(19) United States
(12) Patent Application Publication
(10) Pub. No.: US 2009/0105634 A1
(43) Pub. Date: Apr. 23, 2009

(54) Anodic Reservoir for Electrotransport of Cationic Drug

(75) Inventors: Rama V. Padmanabhan, Los Altos, CA (US); Joseph Bradley Phipps, Sunnyvale, CA (US); Janardhanan Anand Subramony, Belle Mead, NJ (US)

Correspondence Address: Diehl Servilla LLC, 77 Brant Avenue, Suite 210, Clark, NJ, 07066, US

(73) Assignee: ALZA Corporation, Mountain View, CA (US)

(21) Appl. No.: 12/252,731

(22) Filed: Oct. 16, 2008

Related U.S. Application Data
(60) Provisional application No. 60/980,670, filed on Oct. 17, 2007.

(51) Int. Cl. A61N 1/30; H01S 4/00

(52) U.S. Cl. 604/20; 604/501; 29/592.1

(57) ABSTRACT

An electrotransport system for delivery of a cationic drug. The system has a donor anodic reservoir having an insoluble biocompatible polymeric anion source embedded in the reservoir. The anion source has precipitating anions that can precipitate out metal ions generated from sacrificial metal of the anode during electrotransport.

### CROSS REFERENCE TO RELATED APPLICATIONS

The present application claims the benefit of U.S. provisional application Ser. No. 60/980,670, filed Oct. 17, 2007, the entire disclosure of which is incorporated herein by reference.

### TECHNICAL FIELD

The present invention relates to an electrotransport drug delivery system having an anode for driving cationic drugs across a body surface or membrane. In particular, the invention relates to a system having an anodic reservoir for transdermal administration of cationic drug(s) across a body surface or membrane by electrotransport such that the electrotransport does not cause staining on the body surface by metal ion migration.

### BACKGROUND

The delivery of active agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

Many active agents are not suitable for passive transdermal delivery because of their size, ionic charge characteristics, and hydrophilicity. One method for transdermal delivery of such active agents involves the use of electrical current to transport actively the active agent into the body through a body surface (e.g., intact skin) by electrotransport. Electrotransport techniques may include iontophoresis, electroosmosis, and electroporation. Electrotransport devices, such as iontophoretic devices are known in the art, e.g., U.S. Pat. No. 5,057,072; U.S. Pat. No. 5,084,008; U.S. Pat. No. 5,147,297; U.S. Pat. No. 5,395,310; U.S. Pat. No. 5,503,632; U.S. Pat. No. 5,871,461; U.S. Pat. No. 6,039,977; U.S. Pat. No. 6,049,733; U.S. Pat. No. 6,181,963, U.S. Pat. No. 6,216,033, U.S. Pat. No. 6,881,208, and US Patent Publications 20020128591, 20030191946, 20060089591, 20060173401, 20060241548. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the ionic substance to be driven into the body is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

Electrotransport devices require a reservoir or source of the active agent that is to be delivered or introduced into the body. Such reservoirs are connected to the anode or the cathode of the electrotransport device to provide a fixed or renewable source of one or more desired active agents. As electrical current flows through an electrotransport device, oxidation of a chemical species takes place at the anode while reduction of a chemical species takes place at the cathode. Typically, both of these reactions can generate a mobile ionic species with a charge state like that of the active agent in its ionic form. Such mobile ionic species are referred to as competitive species or competitive ions because the species can potentially compete with the active agent for delivery by electrotransport. For example, silver ions generated at the anode can compete with a cationic drug, and chloride ions formed at the cathode can compete with an anionic drug.

In electrotransport or iontophoretic technology, typically, consumable Ag and AgCl electrodes are used at the anode and cathode respectively. The use of consumable electrodes as opposed to the non-consumable platinum or stainless steel electrode has the advantage of mitigating pH shifts induced at the electrode-formulation interface due to electrolysis of water with the latter even at very low voltages.

At the silver anode, during electrotransport, silver is oxidized and, as a result, sliver ion is generated. At the cathode, typically AgCl (solid) is reduced to form metallic silver and chloride ion.

Ag→Ag⁺+e⁻

AgCl(s)+e⁻→Ag^{O}(s)+Cl⁻

At the anode, if silver ions are left to migrate, they can compete with the cationic drug to be delivered and reduce its transport efficiency. Furthermore, silver when allowed to migrate into the tissue of the patient results in a stain on the tissue, which is unsightly. Although the formulation of a cationic drug reservoir with a hydrochloride salt of the drug helps to precipitate some of the silver ions formed in the electrotransport as insoluble AgCl, an excess of the HCl drug salt is needed to ensure that enough chloride is available for interface electrochemistry and to maintain steady state delivery without depletion. However, excessive drug loading could be costly and would increase the potential for drug abuse, particularly if the drug is an opioid.

Furthermore, many drugs are unstable in the HCl salt form and are synthesized as either maleate, citrate or in the acetate form. Electrodes made with other consumable metal would have similar challenges about staining in a similar way. Furthermore, many cationic drugs have risk of being abused. For example, opioids (narcotics) such as fentanyl and its analogs, e.g., remifentanil, sufentanil, alfentanil, lofentanil, carfentanil, trefentanil if delivered transdermally might have higher abuse risk if the amount present in the transdermal device is substantial either before or after prescribed use. Thus, there is a need for transdermal systems containing such drugs with a reduced or minimized drug loading. For the electrotransport of cationic drugs, what is needed is a system with an anodic reservoir that contains less drug than conventional systems and is able to facilitate electrotransport without resulting in staining the tissue.

### SUMMARY

The present invention relates to anodic reservoir for the electrotransport delivery of cationic drugs through a body surface and methods of making and using such anodic reservoirs. This invention identifies features and methodologies to obtain anodic reservoirs for cationic drug delivery in electrotransport applications, which can be done without resulting in metal staining in body tissue. The anodic reservoir includes a precipitate-forming anion source that provides anion to react with metal ion generated from sacrificial anodic metal electrode during electrotransport. The present invention provides anodic reservoirs, electrotransport systems, methods of making and methods of using such anodic reservoirs and electrotransport systems. There are a number of potent drugs that are therapeutic in the cationic form for desired efficacy, e.g., narcotics such as fentanyl salts. These can be delivered iontophoretically with the anodic reservoir of the present invention without staining the tissue, e.g., skin. Further, the anodic reservoir is biocompatible that it would not cause erythema or edema, which skin reactions would make the skin appear to have abnormal color, and thus can be considered to be discoloration.

In one aspect, the present invention provides an electrotransport system for administering an intended cationic drug through a body surface. The system includes an anodic assembly having an anodic reservoir containing the drug and an anodic electrode for conducting a current to drive the drug in the anodic reservoir in electrotransport. The anodic electrode having a sacrificial metal that generates metal ion in electrotransport. The anodic reservoir is in electrical communication to the anodic electrode and contains a cationic drug as well as has an immobile, preferably insoluble, biocompatible polymeric anion source, preferably an anion exchanger. The anion source, e.g., anion exchanger, has precipitate-forming anion that can react with the metal ion to form precipitate in the anodic reservoir, thereby reducing migration of said metal ion to the body. The system also has a cathodic electrode assembly having a cathodic electrode in electrical communication with a cathodic reservoir. A circuitry electrically communicating with the anodic assembly and the cathodic assembly can be used to drive electrotransport of the cationic drug. Preferably the anion exchanger is polysaccharide-based.

In another aspect, the present invention also provides methodology for reducing electrotransport discoloration of skin in electrotransport delivery of a cationic drug. The electrotransport device has an anodic reservoir and an anodic electrode. The anodic electrode has a sacrificial metal that generates metal ion in electrotransport. The anodic reservoir is in electrical communication to the anodic electrode and contains a cationic drug and has an immobile biocompatible polymeric anion source, e.g., anion exchanger, in the anodic reservoir. The anion source has precipitate-forming anion that can react with the metal ion to form precipitate in the anodic reservoir thereby reducing migration of the metal ion to the body. Preferably the device has a designed maximum delivery amount of the cationic drug that is more than 50% of the amount originally present in the device before use. Preferably, the method includes applying an electrotransport device to the skin and using the device to deliver the cationic drug through the skin in an amount up to more than 50% of the amount originally present without discoloring the skin and thereby rendering the device less subject to drug abuse of the cationic drug. Preferably the anion source is polysaccharide-based anion exchanger.

In another aspect, the present invention provides methodology of making anodic reservoirs and electrotransport systems for delivery of cationic drug. To make the anodic reservoir, an immobile anion source having precipitate-forming anion is included in an anodic reservoir. Preferably the anion source is polysaccharide-based. The anodic reservoir also contains a cationic drug, e.g., fentanyl HCl, fentanyl citrate, and the like. The anode contains a sacrificial (consumable) metal, which would generate metal ion during electrotransport. The metal ion and the precipitate-forming anion can react to form an insoluble precipitate. The anode is disposed near and electrically communicates with the anodic reservoir that contains the cationic drug, and is connected to a control circuitry to form an electrotransport system.

In another aspect, the present invention also provides methodology for making anodic reservoirs and electrotransport systems using water-soluble halide (e.g., chloride) source excipients. To make the anodic reservoirs, water soluble quat such as SENSOMER® CI-50 material is included in an anodic reservoir that contains a cationic drug, e.g., fentanyl HCl. An anode is disposed near or on the anodic reservoir that contains a cationic drug and is connected to a control circuitry to form an electrotransport system.

In another aspect of this invention, it is contemplated that the use of anodic reservoir can be useful to deliver non-HCl form of drug with Ag electrochemistry since the precipitation reaction will take place as long the precipitate forming anions and metal ions are present together.

In another aspect of this invention, a kit is provided that contains a device of the present invention and an instruction sheet that instructs a user on the proper way to use the device and describes generally information about the device.

The present invention provides the advantage that metal staining of body tissue due to metal ions migrating to the tissue in electrotransport is prevented or substantially reduced so that no noticeable staining in tissue (e.g., skin) is observed after the period of electrotransport. The metal ion (formed from the sacrificial metal in the electrode) is precipitated out as metal salt precipitate in the anodic reservoir. In the past, excess amount of cationic drug that contains chloride was needed to minimize the amount of silver staining on the skin, see, e.g., U.S. Pat. No. 6,881,208. With the present invention, because the metal ion (e.g., silver ion) is efficiently precipitated out as metal salt (e.g., silver chloride) in the anodic reservoir by precipitate-forming anion included in the insoluble anion source in the anodic reservoir, less drug loading is needed than in the prior systems. Further, with the presence of precipitate-forming anion in the anodic reservoir, even drugs without the same anion or chloride ion can be used in the cationic drug reservoir. The reduction of the amount of cationic drug loading, especially for opioid narcotic drugs such as fentanyl, reduces the risk of the electrotransport drug reservoir being diverted for drug abuse.

Cationic drugs can be effectively delivered without metal staining. For example, at least 100 microgram/cm2hr (i.e., μg/(cm²hr)) of fentanyl base equivalent can be delivered using a current of at 100 microA/cm2 (i.e., mcA/cm² or μA/cm²) without observable silver staining. Using appropriate anodic reservoirs of this invention, no silver staining was observed up to 10 hour, preferably up to 20 hours or more of delivery at current flow of 100 μA/cm². However, there is no reason to believe that if the systems were operated for 24 hours the silver staining results would be different. Thus, such systems can be used for about a day without silver staining.

### BRIEF DESCRIPTION OF THE DRAWINGS

The present invention is illustrated by way of examples in embodiments and not limitation in the figures of the accompanying drawings in which like references indicate similar elements. The figures are not shown to scale unless indicated otherwise.

FIG. 1 illustrates an exploded isometric view of an embodiment of an electrotransport system of this invention;

FIG. 2 illustrates a schematic, sectional view of an embodiment of an electrotransport system showing electrode/reservoir portion of this invention

FIG. 3 illustrates a schematic, sectional view of an electrode placed on a drug reservoir with anion source of this invention;

FIG. 4A shows a representation of the molecular structure of cross-linked dextran as the support in anion exchange material;

FIG. 4B shows a schematic representation of a quaternary ammonium halide source having an exchangeable halide (e.g., chloride) ion;

FIG. 5A to FIG. 5C show the accumulative flux of fentanyl comparing formulations with different fentanyl HCl loading and SEPHADEX™ QAE A-25 loading;

FIG. 6A shows the flux on skin Donor A of fentanyl comparing certain formulations with different fentanyl HCl loading and SEPHADEX™ QAE A-25 loading; and

FIG. 6B shows the flux on skin Donor B of fentanyl comparing certain formulations with different fentanyl HCl loading and SEPHADEX™QAE A-25 loading.

### DETAILED DESCRIPTION

The present invention is related to an anodic reservoir associated in an electrotransport drug delivery system wherein the anodic reservoir contains an immobile, preferably insoluble, polymeric anion source to provide anion to react with a metallic ion to form a precipitate during the electrotransport of the drug. Preferably the metal ions are silver ions generated by the oxidation of metallic silver during the electrotransport process. Thus, staining by the metallic ions migrating to body tissue is substantially reduced or prevented, such that it is not observable visually. The system can be applied to deliver drug to a body surface (e.g., transdermally through skin, or across an ocular tissue, such as conjunctiva or sclera). The anodic reservoir can also be used as counter reservoir for the delivery of anionic drug, in which case the cathode will be the donor side.

The practice of the present invention will employ, unless otherwise indicated, conventional methods used by those skilled in the art in pharmaceutical product development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers. However, when something is said to “include”, “contain”, or “has” a material, it is contemplated that it can be consisted of, or consisted essentially of that material only, unless specified otherwise.

As used herein, the terms “electrotransport,” “iontophoresis,” and “iontophoretic” refer to the delivery of pharmaceutically active agents (charged, uncharged, or mixtures thereof) through a body surface (such as skin, mucous membrane, eye, or nail) wherein the delivery is at least partially induced or aided by the application of an electric potential. The agent may be delivered by electromigration, electroporation, electroosmosis or any combination thereof. Electromigration (also called iontophoresis) involves the electrically induced transport of charged ions through a body surface by electrical potential difference. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, i.e., solvent flow induced by electromigration of other ionic species. During the electrotransport process, certain modifications or alterations of the skin may occur such as the formation of transiently existing pores in the skin, also referred to as “electroporation.” Any electrically assisted transport of species enhanced by modifications or alterations to the body surface (e.g., formation of pores in the skin) are also included in the term “electrotransport” as used herein. Thus, as used herein, the terms “electrotransport,” refer to (1) the delivery of charged drugs or agents by electromigration, (2) the delivery of uncharged drugs or agents by the process of electroosmosis, (3) the delivery of charged or uncharged drugs by electroporation, (4) the delivery of charged drugs or agents by the combined processes of electromigration and electroosmosis, and/or (5) the delivery of a mixture of charged and uncharged drugs or agents by the combined processes of electromigration and electroosmosis. The present invention is especially applicable in the area of electromigration iontophoretic drug delivery.

As used herein, unless specified to be otherwise in content, “distal” refers to a direction pointing away from or being more distant to the body surface, “proximal” refers to a direction pointing to or being nearer to the body surface.

The terms “drug” and “therapeutic agent” mean any therapeutically active substance that is delivered to a living organism to produce a desired, usually beneficial, effect, such as relief of symptoms or discomfort, treatment of disease, or adjustment of physiological functions, e.g., analgesic, regulation of hormone, antimicrobial action, sedatives, etc.

As used herein, the term “fentanyl” generally refers to fentanyl free base and/or fentanyl salt unless specified to the otherwise or the context of its use is clear that it is meant to be otherwise. All fluxes, amounts, or doses of opioids described herein such as those for fentanyl are in free base equivalent (such as fentanyl base) unless specified to be otherwise.

As used herein, the term “matrix” refers to a porous, composite, solid, or semi-solid substance, such as, for example, a polymeric material or a gel, that has pores or spaces sufficiently large for the drug e.g., fentanyl or a pharmaceutically acceptable salt thereof to populate. The matrix serves as a repository in which the drug or its pharmaceutically acceptable salt is contained.

As used herein, the term “immobile” relating to ion source refers to a material that is not driven from the reservoir into the skin in electrotransport by the electrical potential present for delivery of the ionic drug. The ion source can be in particulate form, incorporated into particulates, or being a liquid with large molecular weight in a gel reservoir.

The term “pharmaceutically acceptable sat” refers to salts of a drug, e.g., fentanyl, that retain the biological effectiveness and properties, and that are not biologically or otherwise undesirable.

The term “salt” means a compound in which the hydrogen of an acid is replaced by a metal or its equivalent. As used herein, the salt can be in ionized form in solution or in undissociated form (e.g., in solid form).

As used herein, the terms “transdermal administration” and “transdermally administering” refer to the delivery of a substance or agent by passage into and through the skin, mucous membrane, the eye, or other surface of the body into the systemic circulation.

### MODES OF CARRYING OUT THE INVENTION

The present invention provides an anodic reservoir and an electrotransport system having an anodic reservoir for electrotransport delivery of cationic compounds (e.g., cationic drugs) through a body surface, such as skin or mucosal membrane, e.g., buccal, rectal, behind the eye lid, on the eye such as transconjuctival or transscleral, etc. Electrotransport devices, such as iontophoretic devices are known in the art, e.g., U.S. Pat. No. 5,503,632, U.S. Pat. No. 6,216,033, US20060089591, can be adapted to include cationic drug, e.g., fentanyl or a pharmaceutically acceptable salt therefore, for use for the therapeutic effect. The electrotransport drug delivery system typically includes portions having a reservoir associated with either an anodic electrode or a cathodic electrode (“electrode/reservoir portions”). Generally, both anodic and cathodic portions are present. The electrode/reservoir portion is for delivering an ionic drug or counter ions. The electrode/reservoir portion for the drug reservoir typically includes a drug reservoir in layer form that is to be disposed proximate to or on the skin of a user for delivery of drug to the user. The reservoir can be a matrix that can hold a drug in liquid form, e.g., solution. The drug reservoir typically includes an ionic or ionizable drug. The cationic drug is in the anodic reservoir. The typical iontophoretic transdermal device can have an activation switch in the form of a push button switch and a display in the form of a light emitting diode (LED) or an alpha-numeric display (e.g., LCD) as well. Electronic circuitry in the device provides a means for controlling current or voltage to deliver the drug via activation of the electrical delivery mechanism. The electronics are housed in a housing and typically an adhesive is present on the housing to attach the device on a body surface, e.g., skin, of a patient such that the device can be worn for a few hours to many days, e.g., half day, 1 day, 2 days, 3 days, etc.

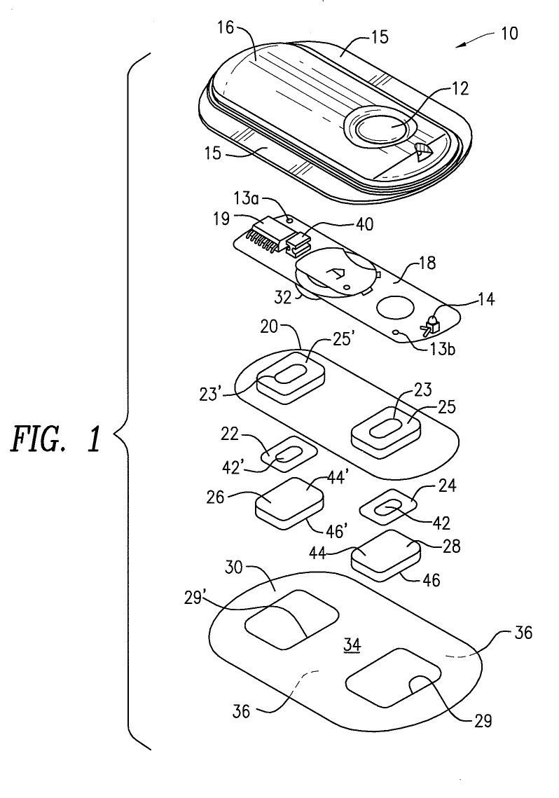

An iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable polymer.

Printed circuit board (PCB) assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10. The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′, 29 in adhesive 30. The skin-facing side 36 of the adhesive 30 has adequate adhesive property to maintain the device on the skin for the duration of the use of the device.

The device for the present invention can be similar to that shown in FIG. 1. The control system, associated with the printed circuit board can be designed in such a way the current and voltage can be controlled for its amplitude, duration, pulsation, wave shape, duty cycles, etc. Methods of designing, fabricating PCB and programming for such implementation are known to those skilled in the art.

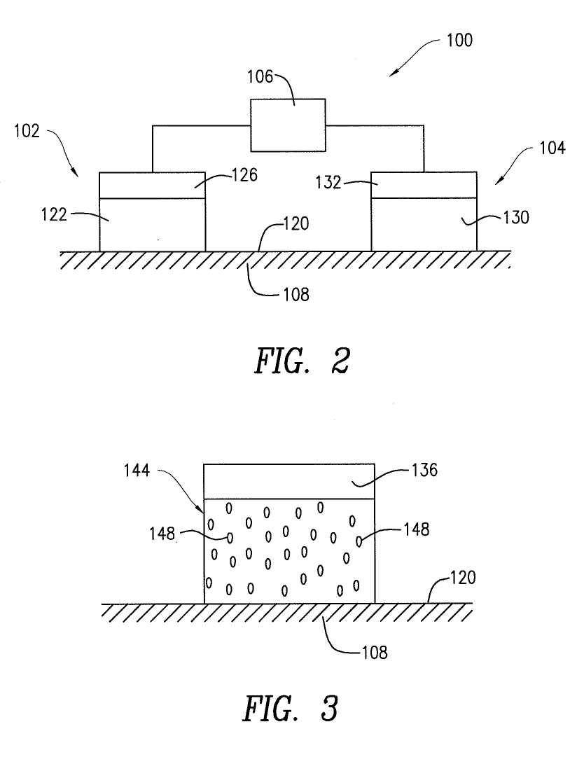

FIG. 2 shows an embodiment of an electrotransport device 100 of the present invention having anode electrode/reservoir assembly 102 and cathode electrode/reservoir assembly 104 connected to and controlled by a controller 106 that provides power source to drive electrical current through the system 100 to the patient tissue 108 through body surface 120 (e.g., skin surface) of the patient. The anode electrode/reservoir assembly 102 has an anodic reservoir 122 contacting the body surface 120 and an anodic electrode 126 disposed on the anodic reservoir 122 that contains chemical reagents (e.g., donor drug) to be delivered to the patient by electrotransport. The cathode electrode/reservoir assembly 104 has cathodic reservoir 130 contacting the body surface 120 and an electrode 132 disposed on the cathodic reservoir 130. The cathodic electrode is the counter electrode if the anodic reservoir contains cationic drug to be delivered. Such a system is useful for iontophoretic delivery of an ionic drug.

The reservoir of the electrotransport delivery devices typically contains a gel matrix (although other non-gel reservoirs, such as spongy or fibrous pads holding liquid, and membrane confined reservoirs, can also be used instead), with the drug solution uniformly dispersed in at least one of the reservoirs. Gel reservoirs are well known and are described, e.g., in U.S. Pat. Nos. 6,039,977 and 6,181,963, which are incorporated by reference herein in their entireties. Suitable polymers for the gel matrix can contain essentially any nonionic synthetic and/or naturally occurring polymeric materials. A polar nature is preferred when the active agent is polar and/or capable of ionization, so as to enhance agent solubility. Optionally, the gel matrix can be water swellable. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent. It is to be understood that the application of the anodes and devices of the present invention is not limited by the reservoir carrier material so long as the reservoir can function to dissociate cationic drug and allow ions to migrate therein. For example, a reservoir that has a semiporous membrane containing a liquid, or a porous pad holding liquid are also applicable for use with an anodic electrode of the present invention. As used herein, gels that contain water are referred to as hydrogels, or simply “gels” sometimes.

In certain embodiments of the invention, the reservoir of the electrotransport delivery system comprises a polyvinyl alcohol hydrogel, as described, for example, in U.S. Pat. No. 6,039,977. Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments of the methods of the invention, is about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise.

To make hydrogels from polyvinyl alcohol (PVOH), polyvinyl alcohol is typically dissolved first (e.g., at 19 wt % in purified water at 90° C. for 30 minutes). Especially useful are the PVOH grades that are well hydrolyzed, e.g., above 80 mol %, preferably 98 mol % or more hydrolyzed, more preferably near 100 mol % hydrolyzed so that there is not many acetyl group left in the PVOH polymer. Minimizing the amount of unhydrolyzed acetyl group left in the PVOH will minimize the release of acetic acid from the PVOH that would tend to lower the pH of the hydrogel. The lowering of pH during storage of the drug reservoir is undesirable since the ionization of the drug, the flux thereof, and irritation on the skin may be adversely affected by pH drift. However, presently available PVOH if 100% hydrolyzed might have too much syneresis (water loss when subject to the freezing process). Thus, 98 to 99.9 mol % hydrolyzed PVOH is preferred. For example, polyvinyl alcohol MOWIOL 28-99, which has 99 to 99.8 mol % hydrolysis, or MOWIOL 10-98, which has 98 to 98.8 mol % hydrolysis (available from KURARAY), can be used. PVOH with more unhydrolyzed acetyl group can also be used. For example, MOWIOL 15-79 has about 79 mol % hydrolysis, MOWIOL 15-96 has about 96 mol % hydrolysis and MOWIOL 26-88 has about 88 mol % hydrolysis. Whereas the back number in the MOWIOL designation represents the extent of hydrolysis, the front number in the MOWIOL represents the viscosity in mPa·s of a 4% aqueous solution at 4° C. For example, MOWIOL 28-99 has a viscosity of 28 mPa·s and MOWIOL 10-98 has a viscosity of 10 mPa·s. The gel solution is then dispensed into molds, and frozen overnight at about −20° C. For example, a useful PVOH solution can have a viscosity of 28 MPa·s (for a 4% aqueous solution at 20° C.). The formed hydrogel is then allowed to imbibe drug as a concentrated aqueous solution at room temperature to obtain the desired drug loading. Alternatively, drug loading is done by adding the drug to the PVOH hydrogel solution before freezing. In the thermally processed formulations, PVOH can be dissolved in purified water at 90° C. as described above. After reduction of the temperature to 50° C., an aqueous solution of the drug is added to the PVOH solution and allowed to mix for 30 minutes. The PVOH-drug mixture is dispensed into molds and freeze-cured. Finished hydrogels were used in flux studies, stability analysis, etc. Similarly, one skilled in the art knows that other forms of reservoirs, made with a material different from PVOH, can similarly be made by forming a reservoir with the drug or imbibing the drug into a formed reservoir matrix.

The present invention provides anions from an insoluble anion source in the anodic reservoir that can form precipitate with the metallic cation generated in the anode during electrotransport. There are a variety of possible electrode electrochemically active component materials and anions associated with drug salts for sacrificial electrode devices that form insoluble salt precipitate. In general, silver, copper and molybdenum metals form insoluble halide salts (e.g. AgCl, AgI, AgBr, CuCl, CuI, CuBr, MoCl₃, MoI₂) and therefore are possible sacrificial anode candidates for delivery of cationic drugs. Insoluble precipitates are formed if the solubility product Kₛₚ of the salt is small, typically less than 1.78×10⁻¹⁰ mol²/kg².

With the anion (e.g., chloride) ion source of the present invention in the anodic reservoir, the anode/reservoir assembly in an embodiment shown in FIG. 2, of course, can be part of an electrotransport system with reservoirs, housing, and other features applicable to a body surface for drug delivery use, similar to those shown in U.S. Pat. No. 6,216,033, and the like.

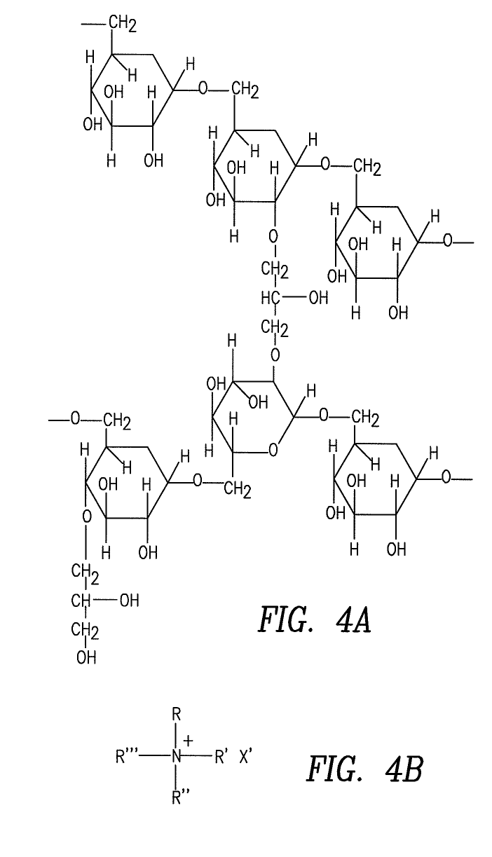

An embodiment of an anodic reservoir with insoluble anion (e.g., halide such as chloride) ion source is generally shown in the schematic illustration of FIG. 3. In FIG. 3, disposed next to the anodic electrode current distributor 136 is an anodic reservoir 144, which includes insoluble anion source 146 embedded within the matrix of the reservoir 144. The insoluble anion source 146 (e.g., chloride source) are preferably particulates on which certain anions (e.g., chloride ions) are associated with the polymeric material therein and can react with metal ions such as silver ions to form insoluble precipitates. Such anions are called precipitate-forming anions because they can form precipitate when reacted with electrode metal ions (e.g., silver ions). These particulates are a source of the precipitate-forming anions. The anion, e.g., chloride ion is associated with or bound to the particulates 146 in an ionic fashion, not covalently, such that the chloride ion can react with silver ion that migrates there, thereby forming silver chloride, which is insoluble in an aqueous medium and therefore will participate out in the anodic reservoir 144. Alternatively, the anion source can be a large molecular weight polymer dispersed in the reservoir.

It is preferred that adequate sacrificial metal (e.g., silver Ag) is present in the anodic electrode, and the surface area is adequate to allow oxidation at an adequate rate to prevent any significant pH drift during electrotransport in which oxidation occurs in the electrode to generate cations. When oxidizable anodic metal is not adequate or the surface is inadequate for forming metal ions during electrotransport, instead of metal being oxidized to form metal cations, water is oxidized in electrolysis, thereby releasing hydronium ions. In electrolysis, gas is also generated. An adequate Ag oxidation would reduce pH drift and the release of gas by electrolysis. Also, competing ions (ions of metal such as silver) are not delivered to the tissue because they are precipitated out (e.g., AgCl).

The anion source for forming precipitate with the metal ions can have a wide variety of anions. Preferably the anion is a halide ion. The preferred anion in the anion source is chloride. In the following, chloride will be used as an illustration for the anion source. It is understood that other halides, such as fluoride, bromide, and iodide can similarly be employed. The precipitate-forming anion source used in the present invention is preferably a macromolecular anion source (e.g., chloride ion source) so that the anions are bound to the macromolecular material that is insoluble and can be held in a layer without diffusion away easily. For example, the anions are bound ionically to solid phase material such as polymeric beads and particulates distributed in the anodic reservoir. The anion source can be a chloride source where the chloride ions are bound to polymeric material, e.g., ion exchange resins with chloride ion as the primary exchangeable ion, or polymeric quaternary ammonium compounds with chloride ions, etc. The polymeric material having bound chloride ions that can react with metal (e.g., silver) ions to form precipitating silver chloride can be an anion exchange material.

Polymeric material having bound anions can be an anion exchanger. Anion exchanger (anion exchange material) can be an organic resin with pendent anionic groups (e.g., SEPHADEX™ QAE resins available from Sigma-Aldrich as a dry powder). Examples of anionic selective materials are described in the article “ACRYLIC ION-TRANSFER POLYMERS”, by Ballestrasse et al, published in the Journal of the Electrochemical Society, November 1987, Vol. 134, No. 11, pp. 2745-2749. An additional appropriate anion exchange material would be a copolymer of styrene and divinyl benzene reacted with trimethylamine chloride to provide an anion exchange material (see “Principles of Polymer Systems” by F. Rodriguez, McGraw-Hill Book Co., 1979, pgs 382-390). Methods for making anion exchange material are known in the art. Typically such methods involve polymerization and cross-linking to produce polymeric material that is insoluble in water. Such ion exchange material can be made into particulates.

For anionic exchange materials of the present invention, strong anionic functionality (such as styrene quaternary ammonium type anion-exchange resin) is particularly preferred. Useful anion sources include polymeric amines and preferred are polymeric tertiary and quaternary ammonium compounds on which anions (e.g., chloride ions) are held ionically and from which the anions (e.g., chloride ions) can react with metal ions (e.g., silver ions) to form precipitate (e.g., insoluble AgCl). More preferred are quaternary ammonium anion exchangers.

Generally, more useful chloride sources include polysaccharide-based materials that can release anions such as halide ions (e.g., chloride ions) to react with metal ions such as silver ions to form precipitates. Such polysaccharide-based polymeric chloride sources have a polysaccharide backbone or a backbone that is derived from polysaccharide. The backbone is therefore a chain containing monosaccharide units, such as glucose, linked by glycosidic bonds. Examples of polysaccharide-based materials that have ionic capacity are SENSOMER® CI-50 from Ondeo Nalco, Naperville, Ill. (which is a cationic starch derivative, i.e., Starch Hydroxypropyltriammonium Chloride) and SEPHADEX™ QAE, a quaternary aminoethyl dextran-based resin crosslinked with epichlorohydrin. SENSOMER® CI-50 is a cationic polysaccharide derived from food grade potato starch that is free of environmental toxic residues. The monosaccharide in starch is glucose. The average molecular weight of SENSOMER® CI-50 is about 2×106 Dalton. It has been reported that no clinically significant responses were seen with SENSOMER® CI-50 material on any of the subjects who participated in a human repeated insult. Tests have shown that SENSOMER® CI-50 was neither a skin irritant nor a skin sensitizer. None of the substances in SENSOMER® CI-50 are listed as carcinogens by the International Agency for Research on Cancer (IARC), the National Toxicology Program (NTP) or the American Conference of Governmental Industrial Hygienists (ACGIH). SENSOMER® CI-50 is biocompatible and has been used in hair products (e.g., shampoo, conditioner) and skin-care products (e.g., cream, lotion). When incorporated into the gel in the reservoir, the SENSOMER® CI-50 is considered to be immobile because of its large molecular weight. Halogen ions such as chloride ions that associate with SENSOMER® CI-50 can react with metal ions such as silver ions to form precipitates. SENSOMER® CI-50 can also be used in conjunction with sacrificial metal (e.g., silver) particles to form particles and can be dispersed and embedded in the anodic reservoir.

SEPHADEX™ ion exchange resins are dextran-based and therefore the monosaccharide in its backbone is also glucose. SEPHADEX™ ion exchange resins are available from Sigma-Aldrich commercially (e.g., in 2007 A.D.). A more preferred material is SEPHADEX™ QAE A-25. Particulate anion exchange material when being formulated into the reservoir typically absorbs aqueous liquid and swells, allowing ion movement and the precipitation by the reaction of metal ions with the anions (e.g., silver ions with chloride ions). It is preferred that in the ion exchanger for the reservoir, before water absorption, has a water uptake capacity of about 10 wt % to 300 wt %, preferably about 20 wt % to 250 wt %. Generally, an anodic electrode is applied to the reservoir to cover 80% to 100% of the surface of the reservoir facing the electrode.

Water soluble halide source such as SENSOMER® CI-50 material can also be used for forming the anodic reservoir in conjunction with consumable metal (e.g., silver) pieces, such as particulates (flakes, particles, beads, etc.). SENSOMER® CI-50 material usually is supplied as a 31-33 wt % dry basis aqueous solution at pH about 3.5-4.5 at room temperature. The soluble halide source can be dispersed with the metal pieces in a solution of the binder dissolved in a solvent. The metal pieces (e.g., Ag) and the halide source can be mixed well in the binder solution and then the solvent is removed from the mixture to render a film with the halide source and the metal (e.g., Ag) pieces embedded in the binder matrix. Water that is in the SENSOMER® material is also evaporated in the drying process. The film can further be divided to form pieces resembling particles. The mixture with the binder solution and metal pieces can further be make directly into particulates and dried. Particle making processes are known to those skilled in the art.

For the anion exchange material that comes in a suspension of solid particles in an aqueous liquid, the particles are removed from the liquid and mixed with the solution that is used for forming the reservoir, e.g., PVOH solution. It is to be understood that the above ion exchange materials may be used in other halide forms.

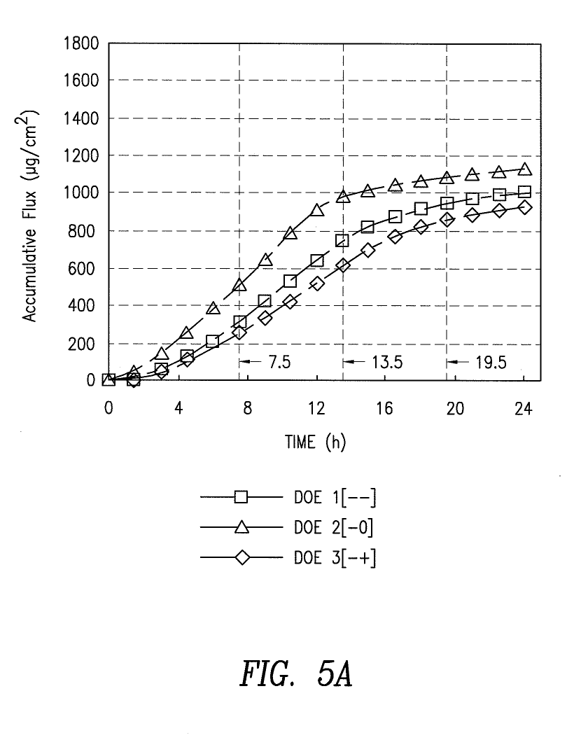

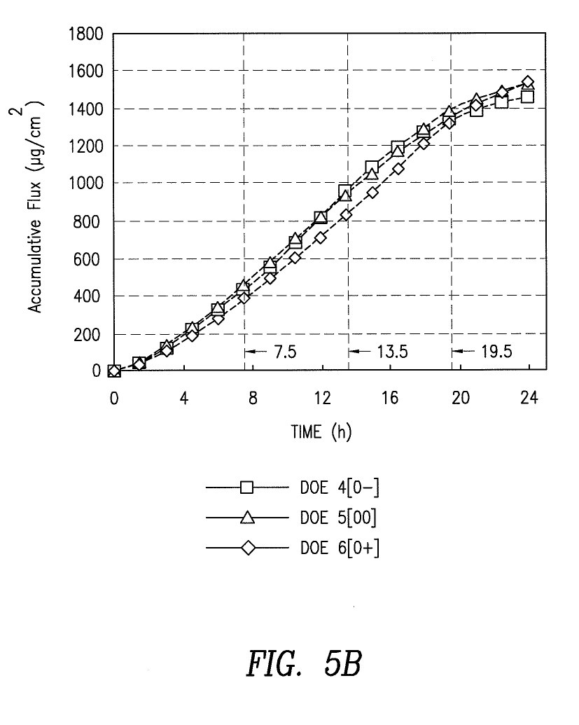

FIG. 4A to FIG. 4B show examples of polymeric anion sources and how they ionically hold on to anions (e.g., chloride ions), which are capable of reacting with metallic ions to form precipitate. FIG. 4A shows the molecular structure of dextran showing the crosslink between two dextran chain units. The crosslinked dextran scaffold can be modified to include functional groups to render anionic or cationic exchanging capabilities. SEPHADEX™ anion exchange resin is an example of a dextran-based resin. SEPHADEX™ QAE A-25 and SEPHADEX™ QAE A-50 have quaternary ammonium functionality on a cross-linked dextran supporting carrier structure. SEPHADEX™ is a dry bead material formed by cross-linking dextran with epichlorohydrin. The SEPHADEX™ QAE A-25 and A-50 are strong anionic exchangers that have about 2.6-3.4 mmol of ionic capacity per gram of dry powder (i.e., with ionic capacity of 2.6-3.4 mmol/g dry basis) and each have particles size range of 40 to 120 microns. The average particle size would be between 40 to 120 microns. The A-25 has more cross-linking than the A-50 and tends to swell less. The pore size of A-25 has about 30,000 Da exclusion limit and the A-50 has about 200,000 Da exclusion limit. The SEPHADEX™ DEAE anion exchanger has weak anion exchange functionality and the resin remains charged and has high capacity at working pH of 2-9. SEPHADEX™ DEAE anion exchanger has about 3-4 mmol of ionic capacity per gram of dry powder. DEAE resins also have A-25 and A-50 varieties. Both QAE and DEAE resins have bead size of about 40 microns to 120 microns. The SEPHADEX™ QAE anion exchanger is a strong anion exchanger and has diethyl-(2-hydroxypropyl)aminoethyl functionalities and is preferred in the present invention. SEPHADEX™ DEAE is 2-(diethylamino) ethyl-SEPHADEX™, i.e., diethylaminoethyl derivative of a cross-linked dextran. Strong anion exchangers are resins that remain charged and have high capacity at working pH of 2-12. For weak anion exchangers, not all the anion exchange functionalities are completely ionized at about pH 2-9. Generally, strong anion exchangers are derived from strong bases and weak anion exchangers are derived from weak bases. Tertiary or quaternary ammonium resin can be useful for anion exchange. Quaternary ammonium resins are especially useful for making strong anion exchangers. Strong anion exchangers, e.g., quaternary ammonium resins, are those anion exchangers that are permanently charged under working pH of 2-10, as understood by those skilled in the art. Preferably the ionic capacity of the dextran based ion exchange has ionic capacity of 2.5 to 4 mmol/g dry basis, more preferably 2.5-3.5 mmol/g dry basis.

FIG. 4B shows a schematic representation of a quaternary ammonium halide source (having a halide X⁻ associated with the quaternary ammonium ion), which halide can react with the metal ion to form a precipitate. It is understood that although the SEPHADEX™ anion exchange resin is used in the Examples herein, other biocompatible strong anion exchange resin can also be used, especially other quaternary ammonium strong anion exchangers (and especially those that are polysaccharide-based biocompatible), since halide ions can be exchanged in similar manners in different anion exchange resins and particulate ion exchange resin can be formulated into a reservoir based on the teaching of the present disclosure. We have found that SEPHADEX™ anion exchangers have the advantage that they are very biocompatible.

A system for delivery of a drug is regulated by competent government drug administration agencies (e.g., the USFDA). An iontophoretic drug delivery system would have an approved nominal drug amount to be delivered, which is the maximum of drug approved by the agency to be delivered by the device, which the device is designed to deliver. The presence of precipitate-forming anion (e.g., chloride) source in the anodic reservoir of the present invention reduces the extent of metallic staining (e.g., silver staining if the electrode contains silver) on body tissue. Generally, the amount of precipitate-forming anion (e.g., chloride) loading in the anodic reservoir is such that substantially all the metal ions (e.g., silver ions) generated by the metal (e.g., silver) during the electrotransport process of the maximum amount to be delivered can be precipitated out so that any metal (e.g., silver) staining of the body surface of the patient is eliminated or reduced to the extent that it is unnoticeable by general visual observation. Thus, the precipitate-forming anion present is adequate to precipitate the metal ions formed for the delivery of the nominal drug amount of the device. It is understood that, however, even if a little reactable precipitate-forming anion (e.g., chloride) present will help to reduce staining due to the metal (silver in the case of a silver-containing electrode) migration. Preferably the anion (chloride ions) loading is such that enough anion (e.g., chloride ion) is present in the chloride ion source stoichiometrically equivalent to the metal ion (e.g., silver ion) that will be generated by the device during the intended period of electrotransport. Of course, more chloride ions than the stoichiometrically equivalent can be present. Since a device is designed to function for a predetermined period of time for a predetermined amount of electrical energy to pass through to deliver a predetermined amount of cationic drug, the stoichiometric equivalent of the metal ion (e.g., silver ion) to be generated can be known and the equivalent amount or more of the anion (e.g., chloride ion) can be included in the anion source before the device is used.

A sufficient amount of solid or polymeric material to which the precipitate-forming anion is bound is present for the loading of anion (e.g., chloride ion) in the reservoir. For example, an adequate amount of anion exchange resin is present for the chloride ions to be held to combine with the stoichiometric equivalent of the silver ions that will be generated in the electrotransport. Knowing the type of anion exchange material being used and the amount of chloride ion loading available (exchange capacity), the right amount of the chloride form of the anion exchange material can be included in the anodic reservoir chloride source. Knowing the type of anion exchange material to use, one skilled in the art can readily calculate, as well as experimentally determine the amount of the ion exchange material to use in the anodic reservoir. Further, anions other than chloride, such as other halides, can similarly be employed by those skilled in the art based on the present disclosure.

In a hydrated hydrogel of PVOH, preferably the amount of the insoluble anion source is about 1 wt % to 4 wt % (considering the insoluble anion source before fluid absorption in forming the hydrogel). More preferably the insoluble anion source is about 1 wt % to 2 wt %. In a preferred mode in which the anion source is SEPHADEX™ QAE strong anion exchanger (e.g., QAE A-25 or A-50), preferably the ion exchanger is less than about 4 wt %, more preferably about 1 wt % to 2 wt %, even more preferably 1 wt % to 1.5 wt %, and especially preferably about 1.2 wt % to 1.3 wt % in the anodic reservoir. Preferably the PVOH is of a grade that is 98 mol % or more hydrolyzed, such as MOWIOL 28-99 or MOWIOL 10-98.

A wide variety of ion exchange resins are available commercially. Methods for making ion exchange resins and films are known in the art. See, e.g., pages 52-55 of “A First course in ion permeable membranes”, T. A. Davis, J. D. Genders, D. Pletcher, The electrochemical consultancy, England, 1997. For example, a composition having poly(vinylchloride), styrene, divinylbenzene, 4-ethylbenzene, 2-methyl-5-vinylpyridine, benzoyl peroxide, and dioctyl phthalate are mixed into a paste. The composition is heated at about 350-390° K. to polymerize and form a layer. The anionic exchange functionalities are then introduced by reacting the layer with suitable agents. For example, the polymerized layer can be soaked in 50:50 chloromethyl methyl ether” carbon tetrachloride containing 5 vol % SnCl₄ at 283° K. to introduce chloromethyl groups and then quarternizing by treatment with a trimethylamine solution. Alternatively, to introduce the chloromethyl group, chloromethyl styrene can be included as one of the monomers in the polymerization reaction, before the quaternization. An alternative method of making anion exchange layers involves including vinylpyridine as one of the monomers and following up the polymerization with quaternization using a solution of methyl iodide in petroleum ether.

Because the anion source in the anodic reservoir precipitates out metal ions generated in the anode, the system is applicable to additionally deliver cationic drugs, including a wide variety of drug as long the drug is cationic and can be included in a reservoir to be delivered iontophoretically. Cationic drugs that can be delivered include analgesics, antitumor drugs, antibiotics, histamines, and hormones. Examples of cationic drugs that can be delivered include, e.g., amiloride, digoxin, morphine, procainamide, quinidine, quinine, ranitidine, triamteren, trimetoprim, or vancomycin, procain, lidocaine, dibucaine, morphine, steroids and their salts. For example, hydrochloride salts of vancomycin, procain, lidocaine, dibucaine, and morphine, and acetate salt of medroxyprogesterone are drugs having cationic moieties that can be delivered. Examples of analgesic drug that can be delivered include narcotic analgesic agent and is preferably selected from the group consisting of fentanyl and functional and structural analogs or related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimepheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine. For more effective delivery by electrotransport such as iontophoresis, salts of such analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, halides (such as chloride, bromide, iodide), citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxybutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. It is known in the art that halide salts are in the form of acid halide for many of such salts (e.g., hydrochloride). The more preferred salt is hydrochloride. Such salts can become ionized in aqueous environment and the cation can be delivered to produce physiological effect on the patient. For example, opioid salt will form opioid cation, e.g., fentanyl HCl salt will form fentanyl cation.

The rate of delivery of fentanyl (i.e., fentanyl HCl) has been investigated and described before, e.g., in U.S. Pat. No. 6,216,033, and the method and rate of delivery (i.e., the current and flux) of such description can be adapted for the present invention. It has been determined that a transdermal electrotransport dose of about 20 μg (microgram) to about 60 μg of fentanyl (base) equivalent, delivered over a delivery interval of up to about 20 minutes, is therapeutically effective in treating moderate-to-severe post-operative pain in human patients having body weights above about 35 kg. Preferably, the amount of fentanyl delivered is about 35 μg to about 45 μg over a delivery interval of about, 5 to 15 minutes, and most preferably the amount of fentanyl delivered is about 40 μg over a delivery interval of about 10 minutes. Since fentanyl has a relatively short distribution half life once delivered into a human body (i.e., about 3 hours), the method of inducing analgesia preferably includes a method for maintaining the analgesia so induced. Thus the method of transdermally delivering fentanyl by electrotransport preferably includes delivering at least 1 additional, more preferably about 10 to 100 additional, and most preferably about 20 to 80 additional, like dose(s) of fentanyl over subsequent like delivery interval(s) over a 24 hour period. A current of about 150 μA to about 240 μA can be used. Adequate fentanyl salt (e.g., fentanyl HCl) is loaded into the anodic reservoir before the device is used, e.g., for 1 day delivery.

It has been shown that to ensure the fentanyl flux does not decrease substantially the fentanyl loading at the start of the electrotransport needs to be maintained above about 11 mM, and preferably above about 16 mM. For fentanyl HCl, the 11 to 16 mM concentration is equivalent to about 4 to 6 mg/mL. Other fentanyl salts (e.g., fentanyl citrate) will have slightly differing weight based concentration ranges based on the difference in the molecular weight of the counter ion of the particular fentanyl salt in question. Further, to ensure silver is not deposited in the skin causing discoloration (transient epidermal discoloration, TED) in electrotransport, the fentanyl loading at the start of the electrotransport needs to be at least double the amount the device is designed to deliver. See U.S. Pat. No. 6,881,208. This was the approach taken in the traditional IONSYS system (Ortho-McNeil Inc., Raritan, N.J.). Devices for delivery of pharmaceuticals, especially opioids, are carefully regulated by government agencies. Every device of such kind needs to seek governmental approval by stating the amount of the specific the device is to deliver. The amount of a drug to be delivered by a regulated device is therefore publicly known and easily ascertained. Information about the drug device is generally available in the form of a physician or patient package insert or label.

In the traditional approach, in the specific case of an electrotransport delivery device having a polyvinyl alcohol based donor reservoir containing fentanyl hydrochloride and having a total weight (on a hydrated basis) of about 0.3 to 0.8 g, which device (1) has an anodic donor electrode comprised of silver (e.g., silver foil or silver powder-loaded polymer film) which is in electrical contact with the donor reservoir, (2) has an electrical power source which applies a DC current of about 100 μA to 230 μA to the donor and counter electrodes, (3) applies a current density, measured as the total applied current divided by the skin contact area of the donor reservoir, of about 60 μA/cm2, and (4) is capable of applying such current for up to about 80 separate delivery intervals of about 8-12 minutes duration, the fentanyl HCl loading needed to induce and maintain analgesia is about 2.5 to 3.5 mg, yet the fentanyl HCl loading needed to prevent TED is at least about 8 to 10 mg, and preferably at least about 11 to 13 mg. More specifically in the case of an electrotransport delivery device having a polyvinyl alcohol based donor reservoir containing fentanyl hydrochloride and having a total weight (on a hydrated basis) of about 0.5 to 0.8 g, which device applies a DC current of about 170 μA to the electrodes, and is capable of applying such current for up to about 80 separate delivery intervals of about 10 minutes duration, the fentanyl HCl loading needed to induce and maintain analgesia is about 3 mg, yet the fentanyl HCl loading needed to prevent TED is at least about 9 mg, and preferably about 12 mg which in a hydrogel of about 2.7 cm² in area and about 3/32 in (2.4 mm) thick, the loading is about 3 mg/cm² to 6 mg/cm². The fentanyl HCl loading in IONSYS system is about 10.8 mg fentanyl free base equivalent in 600 mg PVOH gel for delivery of about 3.2 mg fentanyl free base equivalent maximum.

Generally a drug delivery device is approved by a competent national drug administration authority rated for a maximum delivery amount. For example, the IONSYS system was authorized by the USFDA to deliver a maximum of 80 doses of 40 μg per dose. Thus, the IONSYS system was designed and approved by drug administration authority to deliver a maximum amount of 3200 μg of fentanyl base equivalent. The IONSYS system can be said to have a nominal maximum delivery of 3200 μg of fentanyl base equivalent. However, in the present invention, with the incorporation of insoluble anion source in the anodic reservoir, the amount of cationic drug loading can be reduced and still deliver the amount of the drug for which the device is designed and approved and prevent epithelial discoloration due to silver migration to the skin. Preferably, the amount of drug (e.g., fentanyl HCl) loading in the anodic reservoir is less than double the amount of drug the system is designed to deliver at a maximum. For example, if the device is designed to deliver 3200 μg of fentanyl at maximum, the device contains less than 6400 μg of fentanyl (correspondingly the equivalent amount of fentanyl HCl) and still does not cause skin staining. At the end of the delivery of a maximum amount of the drug, the drug remaining in the anodic reservoir is preferably 50% or less, preferably less than 50%, more preferably 40% or less, even more preferably 30% or less of the drug amount originally present in the electrotransport system at the start. Thus, although more fentanyl loading can be used, preferably, to reduce fentanyl abuse risk, fentanyl loading is 200% or less of the maximum amount of fentanyl designed to be delivered by the device. In the anodic reservoir before electrotransport, preferably the fentanyl salt concentration in the liquid in the hydrogel is less than 0.03 mM, more preferably about 0.15 mM to 0.25 mM, more preferably about 0.15 mM to 0.2 mM for a system that delivers the same amount of fentanyl as IONSYS. Thus, in the anodic reservoir of the present invention, the concentration in the liquid in the reservoir is less than that in the traditional system (see IONSYS system, which has 0.03 mM fentanyl HCl). Therefore the electrotransport system of the present invention poses a smaller risk of being abused.

Incorporation of the drug solution into the gel matrix in a reservoir can be done in any number of ways, i.e., by imbibing the solution into the reservoir matrix, by admixing the drug solution with the matrix material prior to hydrogel formation, or the like. Hydrogels can be made with standard methods known in the art, e.g., PVOH gels can be made with freeze-thaw cycles from a PVOH solution. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by on skilled in the art.

General methods of making gels for reservoirs and incorporating drugs in the gels are known in the art. General methods for making electrodes, printed circuit boards, adhesives, housing, and other kind of iontophoretic device components are known. General methods for making electrotransport devices from their components are known in the art. Generally, components such as the reservoirs, the electrodes, the printed circuit boards, the housing parts, adhesive, displays are made and then the components are assembled by connecting the electrical connections and affixing the separate pieces together. For example, the reservoir gel can be laid into a depression in the lower housing to contact the electrode and the lower housing is fitted with the upper housing to enclose the printed circuit between the lower and upper housing. An adhesive, protected by a peelable release liner, is laid on the lower housing to provide adhesion when the device is to be used.

General methods of using electrotransport devices are known in the art. Generally, a user, such as a patient, more often a care giver (e.g., doctor, nurse, etc.) will open a package pouch, remove the device from the pouch, check the device for proper functioning, remove the peelable protective release liner and apply the device on the body surface of the patient for the device to adhere thereto. During the use period, the control button on the device is manipulated to control the delivery of doses of the drug and display of information. Of course, the use of the gel and reservoir material of the present invention is applicable to a wide variety of other components of the device and is not dependent on the specific type of construction material of the other parts of the device, e.g., the control circuit, etc.

The electrotransport devices of the present invention can be included in a kit that contains the device and includes an instruction print, such as an insert or printings on a container, and the like that provides instruction on the how the device is to be applied to a patient and how often the device can be activate and the maximum amount of drug the device is designed to deliver, etc. The instruction of use can include method of activating the device and determining the doses and amount of drug already delivered. The instruction of use can also include brief description of the drug, the construction of the device, pharmacokinetic information, information on disposing the device that contains a control substance (e.g., fentanyl) and warnings.

### Biocompatibility of SEPHADEX™ Resin

In electrotransport in which a drug reservoir is in contact with the body surface, e.g., skin, for hours, e.g., 20 hours, 24 hours, or more, it is important that the material in the drug reservoir is biocompatible with the body surface, e.g., skin. Certain matrix material such as PVOH has been shown to be biocompatible in the art and is already used in iontophoretic devices. However, suitable biocompatible anion exchanger has not been found, especially for strong anion exchanger. We have found that dextran-based strong anion exchanger resins, such as SEPHADEX™ QAE resin, to be biocompatible, in that the extracts of such resins do not cause adverse reaction in skin, and therefore would not be expected to cause inflammation, erythema or edema when matrix with such resins are deployed on skin for electrotransport. Inflammation, erythema or edema can be considered to cause discoloration of skin since they cause abnormal appearance, especially in color on the skin.

SEPHADEX™ QAE A-25 resin was extracted with four extraction vehicles:1) 0.9 wt % sodium chloride USP solution (SC); 2) ethanol in saline 1:20 solution (AS); 3) polyethylene glycol 400 (PEG); and 4) cottonseed oil, NF (CSO). The extractions were made at a ratio of 2 g resin to 20 ml vehicle at 50° C. for 72 hours with pH adjusted to 7 with sodium hydroxide if necessary. The resin particles were filtered off to obtain the extracts. Mice were weighed and five mice were each injected either intravenously or intraperitoneally with each test extract at a dose of 50 ml/kg of extract (SC, AS, or CSO) or 10 g/kg of PEG extract. The corresponding extraction vehicles without extracting from the ion exchanger were also injected into control mice as controls. For PEG, the PEG extracts and control blanks were diluted with saline to make 0.2 g of PEG/ml, which corresponded to injection volume of 50 ml/kg. The mice were observed for adverse reactions such as convulsions or prostration, weight loss or death. The result showed that weight data were acceptable, there was no mortality, and the mice injected with the extracts appeared normal, without unexpected events. The ones injected with AS extracts appeared similar to those in the AS control as there may be lethargic effect caused by ethanol from the vehicle. Therefore, there was no evidence of toxicity with the test extracts.

SEPHADEX™ QAE A-25 extracts for SC, AS, PEG and cottonseed oil were used at 2 g ion exchanger to 20 ml vehicle similar to the above. The PEG extracts and control blanks were diluted with SC vehicle to make 0.12 g of PEG/ml. New Zealand white female rabbits were tested with intracutaneous injection with the extracts and controls. Each test rabbit was injected with 0.2 ml of test extract or the corresponding vehicle. Observation for erythema (ER) was conducted for 72 hours with rating scale of 0 to 4, wherein 0 means no sign of erythema, 1 means barely perceptible color change, 2 means a well defined pink color, 3 means moderate to sever redness, and 4 means severe redness (beet red) to slight eschar formation. Observation for edema (ED) was conducted for 72 hours with rating scale of 0 to 4, wherein 0 means no sign of edema, 1 means barely perceptible edema, 2 means a slight well defined area of swelling, 3 means moderate edema with raised about 1 mm, and 4 means severe edema (raised more than 1 mm and may extend beyond the area of exposure). The result showed that for SC, AS, and PEG the ED and ER were all 0. For the CSO extracts, the extract results and control results were the same, with a score of 2 for ER and a score of 1 for ED. Thus, the rabbit ER and ED tests showed that SEPHADEX™ QAE was biocompatibility and would not cause ER, ED or skin physiological color change due to inflammation in the skin (in other words, discoloration due to such skin changes).

Further, test results of the effect of test extract in vitro on lymphocyte proliferation (stimulation index) and cytotoxicity (IC₅₀) on HELA cells showed that SEPHADEX™ QAE resins are nontoxic and nonmitogenic. Extracts of ion exchange resins were generated from powder based polymers under passive (aqueous) conditions. The materials were examined for their mitogenic and cytotoxic activities. Mitogenicity tests were performed using in vitro lymphocyte proliferation assays. Cell cytotoxicity was assessed using MTT and LDH release assays. Mitogenicity testing was performed on lymphocytes obtained from mice, guinea pig, rat, and humans. Human fibroblasts and HELA cells were used for cytotoxicity testing. Cholestyramine resin (C1734 Cholestyramine Resin, USP from Spectrum Chemicals, Gardena, Calif., USA) was also tested similarly for comparison.

### Preparation of Passive Resin Extracts

Ten mL of RPMI-1640 culture medium (containing penicillin/streptomycin) was added to one gram of the test resin (dry form) and placed in a 50 mL conical tube. The solution was placed on a circulating rotator (slow speed rotator) for 72 hours at room temperature. Thereafter, extracts were obtained by centrifuged at 500 g (10 min). The supernatant was collected and sterile filtered through 0.22 μM filter and stored frozen (−20° C.) as extract till use. The remaining pellet was discarded. These extracts were tested for biocompatibility by tests for mitogenic activity with lymphocytes and on cytotoxicity.

Isolation of Lymphocytes from Mouse Spleen or Lymph Nodes

Lymph nodes (axillary, brachial, inguinal, popliteal, and cervical) and/or spleens from euthanized animals were removed under aseptic conditions and placed in sterile tube containing PBS, or similar media. The tissues were then teased to release the cells. Cells were filtered, centrifuged, washed and separated with standard procedures known in the art to separate lymphocytes. Cell counts were determined using a hemocytometer and viability was assessed using trypan blue. The cells were resuspended to a final concentration of 2-3×10⁵ cells/mL (10% FBS final concentration in culture).

Isolation of Lymphocytes from Rat or Guinea Pig Spleen

Spleens from euthanized animals were removed under aseptic conditions and placed in sterile tube containing PBS, or similar media. The tissues were then transferred into a sterile Petri dish containing cell culture media. Cells were released by teasing the tissue cells with forceps and syringe/needle. Cells were filtered, centrifuged, washed, over Lympholyte-M (room temperature), and separated with standard procedures known in the art to separate the lymphocytes with procedures known in the art. Cell counts were determined using a hemocytometer and viability was assessed using trypan blue. The cells were resuspended in culture medium to a final concentration of 2-3×10⁵ cells/mL.

Isolation of Guinea Pig Lymphocytes from Peripheral Blood

Blood was collected from guinea pigs under sterile conditions into sodium citrate tubes. Blood was diluted 1:1 with 1×DPBS (1% penicillin/streptomycin) into sterile polypropylene tubes. The cells were then layered blood over Lympholyte-M (room temperature) and separated out the lymphocyte cells with procedures known in the art and similar to the above.

Isolation of Human Lymphocytes from Peripheral Blood

Human blood was collected under aseptic conditions by venipuncture into sterile heparinized tubes. The blood was transferred to sterile 50 mL polypropylene tubes and diluted 1:1 with 1×DPBS containing 1% penicillin/streptomycin. The diluted sample was carefully layered Histopaque-1077 separation media (adjusted to room temperature). The samples were then centrifuged for 20 minutes at 400 g. After centrifugation, the lymphocytes were collected at the interface and transferred to 50 mL tubes. The suspension as adjusted to about 35-40 mL with 1×DPBS with 0.1% BSA (adjusted to 4° C.) and centrifuge at 400 g for 10 minutes. The supernatant was discarded. Removal of residual red blood cells present in the pellet was accomplished by the addition of 4.5 mL of sterile deionized water and resuspension of the cells. Shortly thereafter, 0.5 mL of 10×DPBS was added in order to restore isotonic conditions. Culture medium was then added. The cells were resuspended to 3.0×10⁶ cells/mL in RPMI cell culture medium (final serum concentration in culture is 5% NHS).

### Lymphocyte Proliferation Assay

For each sample, 100 μL of PBL (3.0×10⁶ cells/mL) were dispensed into a 96-well round-bottom plate (3.0×10⁵ cells/well). To this, 100 μL of media containing appropriate reagents (e.g., test antigens or controls), i.e., extract, were added to bring the final volume to 200 μL/well (depending on cell type, either 10% FBS or 5% NRS or 5% NHS). Replicate wells (at least triplicates) were established for each variable. Cells were maintained in a tissue culture incubator (37° C., 5% CO₂). Twenty four hours after culture initiation (day 1), the cell were pulsed with 1 μCi of ³H-thymidine (20 μt/well, 50 μCi/mL stock). On Day 2 (18-24 h after pulse), cells were harvested using cell harvester (Packard GF/C plates). After harvesting, GF/C filter plates were allowed to air-dry. The underside of the GF/C plates were sealed with an adhesive, and 20.5 μl of MicroScint-20 is added to each well. A seal (TopSeal) was placed on to cover the top of plate. ³H-thymidine incorporation was determined by β scintillation counting (Packard TopCount). Cultures were evaluated for 48 to 72 hours. As a measure of cellular proliferation, the results were expressed in counts per minute (CPM). Each variable was evaluated in at least triplicates, and the results were calculated as average CPM+/−the standard error of the mean (SEM). Lymphocyte proliferative responses to the test compounds were compared to cell cultured in media alone (i.e. background). The data were also expressed as stimulation index (SI) and were calculated from:

$$S I = \frac{average C P M for stimulated wells}{average C P M from unstimulated control wells}$$

A response is considered positive if the SI value is >2.0, and the response is dose dependent.

### MTT and LDH Cytotoxicity Assays

In the assays, suspensions of 2.0×104 cells were added per well in a flat-bottomed 96-well plate. Cells were allowed to adhere to the plate overnight. Thereafter, the media was removed, and 200 μL of test solution (i.e., resin extract) was added per well. Test solutions were incubated with cells for 20 hours. After incubation, the supernatants were collected and used for LDH release (Lactate Dehydrogenase Release) assay. The MTT assay ((3-[4,5-dimethylthiazol-2-yl]-2,5-diphenyl tetrazolium bromide) assay) was performed on the adherent cells. MTT assay and LDH release assay are well known in the art of cytotoxicity evaluation.

### Results

MTT and LDH release assays were performed for each of the extracts obtained above. SEPHADEX™ QAE showed no cytotoxicity. In contrast, USP grade cholestyramine resin showed cytotoxicity because the 50% inhibitory concentration for (IC₅₀) cholestyramine was found to be at a 1:18.5 dilution. There was no mitogenic activity in lymphocytes cultured with SEPHADEX™ QAE. There was no significant mitogenic activity with SEPHADEX™ QAE in any of the tests. Mouse (strain: Balb/c) lymphocytes demonstrated a positive lymphocyte response to cholestyramine (stimulation index=14-33). Guinea pig lymphocytes, isolated from peripheral blood or spleen, showed no reactivity to cholestyramine. Rat lymphocytes, derived from spleen cells, showed positive lymphocyte activity towards cholestyramine (stimulation index=3.9). Human peripheral blood mononuclear cells (PBMC) showed no activity towards cholestyramine.

Also, tests to show histamine release from mouse mast cells (cell line 10P2) showed that when the cells were cultured with SEPHADEX™ QAE A-25 resin extract there was no increase in histamine release. Thus, all the evidence indicated that SEPHADEX™ QAE resin caused no adverse biocompatibility reaction at all. From our experimental results we found that the SEPHADEX™ QAE strong anion exchanger is exceptionally biocompatible, considering that we have found even USP grade cholestyramine resin is not as biocompatible as the SEPHADEX™ QAE ion exchanger.

### EXAMPLES

### Example 1 Preparation of Hydrogel Reservoir

Hydrogels were typically prepared by dissolving polyvinyl alcohol (PVOH) (MIWOIOL 28-99) at about 19 wt % in purified water at 90° C. for 30 minutes. The material was heated until the PVOH went into solution. The temperature was lowered to about 60° C. and maintained for around 30 minutes until solution was free of air bubbles. The PVOH solutions were pH adjusted to about 4.5 and the resulting mixtures were poured into molds to be cured by freezing. The PVOH gels were made at 3/32 inch (2.4 mm) thickness. During the making process, the uncured materials were covered with a temporary liner to prevent particle contamination and reduce water loss. The poured materials in the molds were cured by a freeze-thaw cycle process (64 minute time-temperature cycle with a freeze temperature of −25° C. or below). The process involved at least one cycle. Typically one freeze/thaw cycle was used, although the freeze/thaw cycle could be done twice or repeated many times if desired. The thawing was typically done about 5° C. After the freeze/thaw cycling, the gels would acquire enough strength for the temporary liner to be removed easily because the “cross-linking” of the PVOH in the gel removed the tackiness from the surface of the gel. Since no cross-linking agent was used, the cross-linking was not a true covalent cross-linking but rather an interaction of the PVOH chains, reversible by heating. The degree of cross-linking was estimated in terms of dynamic modulus. A lower limit of 1400 to 1700 Pa was set to be acceptable, but no upper limit was set. For the gels that were to include a drug, the formed hydrogels were then allowed to imbibe a drug solution at room temperature to obtain the desired drug loading. The drug was imbibed overnight. Alternatively, drug loading could be achieved by adding the drug to the PVOH solution before freezing. The gels after imbibing were punched to 1.27 cm² circular size. With a similar process, PVOH hydrogel that contained SEPHADEX™ ion exchanger were made by mixing powder-like SEPHADEX™ (e.g., QAE-25 available from Sigma-Aldrich as a dry powder) ion exchanger resin particles in chloride form into PVOH solutions in amounts to make gels of the desired wt % of SEPHADEX™ ion exchanger resin and wt % PVOH.

The dynamic moduli (a measure of complex moduli) of SEPHADEX™ containing PVOH gels were evaluated to characterize mechanical properties of the gels. Complex modulus is defined as the ratio of the amplitude of the sinusoidal stress to the strain at any given time, t, and angular load frequency, ω. Mathematically, the dynamic modulus is defined as the absolute value of the complex modulus, or (σo/εo), where σo is the peak (maximum) stress and ε0 is the peak (maximum) strain. Dynamic modulus is thus the ratio of stress to strain under vibratory conditions (calculated from data obtained from either free or forced vibration tests, in shear, compression, or elongation). The standard method of measuring dynamic moduli of gels was used. Testing instrument such as DMA 2980 (TA Instruments, New Castle, Del.) or similar equipment could be used. For example, we used Haake RS 100 rheometer with MV3 sensor system at 1 Hz at 25° C. The gel specimen was held between two parallel plates and submected to a sinusoidal oscillation. We found that dynamic modulus was nonlinearly affected by the frequency of oscillation. We tested the gels with a frequency of 0.1 Hz to 1 Hz, under which the dynamic modulus was stable. It has been determined that a dynamic modulus above 1400 Pa is satisfactory for a hydrogel for a reservoir of the present invention. A PVOH gel with complex modulus of about 20000 Pa is still useful. Since the weight percent of fentanyl in the hydrogel formulations is minimal compared to the formulation mix, fentanyl HCl was excluded from the formulation while making the gels for dynamic modulus studies. The complex modulus (represented by dynamic modulus) results are summarized in the Table 1 below. The control gels were cathode hydrogel with the same PVOH composition but without SEPHADEX™ resins. The PVOH grade was PVOH MOWIOL 28-99.

TABLE 1
Complex Modulus of PVOH with SEPHADEX QAE A-25

| Sample ID | 0.1 Hz | 1 Hz | 10 Hz | Age |
|---|---|---|---|---|
| S1 | 3780 | 4030 | 4490 | 5 days old |
| S2 | 3930 | 4150 | 4899 | 5 days old |
| S3 | 3840 | 4060 | 4750 | 5 days old |
| Average | 3850 | 4080 | 4680 | 5 days old |
| Cathode control | 3750 | 4160 | 4700 | 2 years |

Table 1 shows the complex moduli in Pa. From the table it is clear that the dynamic moduli for the SEPHADEX™-containing gels (5 days old) are higher and equivalent to a two year old cathode control formulation. Additional tests showed that 23 wt % PVOH gels containing 5 wt % SEPHADEX™ were found to be stiffer than the regular 23 wt % PVOH gels. However, from other viscosity experiments, it appeared that formulations with 23 wt % PVOH and 5 wt % SEPHADEX™ would produce a gel with viscosity too high for the manufacturing process.

### Comparison of PVOH for Hydrogel

Hydrogels were made with MOWIOL 28-99 (“PVOH 28-99”) and MOWIOL 10-98 (“PVOH 10-98”) with SEPHADEX™ QAE A-25 and the complex moduli were measured. The data are shown in Table 2. The IONSYS Placebo (control) was a gel from an IONSYS system. The results showed that useful hydrogels can be made with either PVOH 10-98 or PVOH 28-99. The hydrogels with PVOH 10-98 tend to have lower complex moduli than those with PVOH 28-99 having the same PVOH contents and SEPHADEX™ contents.

TABLE 2
Complex Moduli of Hydrogels

| | | 10 rpm @ |
|---|---|---|
| Sample | 10 rpm @ 60° C. | 85° C. |
| 20% PCOH 10-98 | 587 | 319 |
| 23% PCOH 10-98 | 1250 | 635 |
| 26% PVOH 10-98 | 2415 | 1184 |
| IONSYS Placebo 9061-017 SD/RT | 8847 | 4417 |
| 20% PVOH 10-98 + 1.3% Sephadex | 1356 | 690 |
| 23% PVOH 10-98 + 1.3% Sephadex | 2835 | 1317 |
| 19% PVOH 28-99 + 1.3% Sephadex | 20710 | Not Tested |
| 23% PVOH 28-99 + 1.3% Sephadex | 78280 @ 5 rpm | Not Tested |

Table 3 below is a table listing a design of experiment (DOE) study for different PVOH contents and SEPHADEX™ contents. The column on design model indicates the variations (−, 0, +) of the parameters in the table being evaluated relative to the neighboring formulations, in which the left symbol represents the left parameter and the right symbol represents the right parameter. The + means high lever, the − means low level, and the 0 means mid level of an ingredient being considered. In each formulation 0.18 wt % of cation exchange resin POLACRILIN IRP 64 in sodium form was included for pH buffering. Table 4 shows the complex moduli of the gels of Table 3, as dynamic moduli measured by Haake RS 100 at MV3 at 1 Hz at 25° C. The results showed that PVOH 10-98 can be employed up to 23 wt % with SEPHADEX™ up to 5 wt % and the complex moduli were still about or below 20000 Pa. Even with 28 wt % PVOH 10-98, the complex modulus was still only about 13400 Pa if the SEPHADEX™ content was 1.3 wt %.

TABLE 3
Gel Formulations Prepared for DOE Study

| Formu- | | Designed | | Prepared | |
|---|---|---|---|---|---|
| lation | Design | PVOH | SEPHADEX | PVOH | SEPHADEX |
| # | Model | 10-98 | QAE A-25 | 10-98 | QAE A-25 |
| 1 | −− | 19% | 1.3% | 18.21% | 1.32% |
| 2 | −0 | 19% | 3.2% | 18.99% | 3.19% |
| 3 | −+ | 19% | 5.0% | 19.47% | 5.12% |
| 4 | 0− | 23% | 1.3% | 22.86% | 1.30% |
| 5 | 00 | 23% | 3.2% | 23.65% | 3.28% |
| 6 | 0+ | 23% | 5.0% | 23.35% | 5.07% |
| 7 | +− | 28% | 1.3% | 27.27% | 1.27% |
| 8 | +0 | 28% | 3.2% | 27.27% | 3.13% |
| 9 | ++ | 28% | 4.0% | 27.87% | 4.00% |
| 10 | N/A | 19% | 0 | 18.81% | 0.00% |
| | | (PVOH 28-99) | | | |
| 11 | N/A | 28% | 5.0% | 28.66% | 5.10% |

TABLE 4
Complex Modulus (G*) @ 1 Hz/25° C., Measured by Haake RS100 at MV3
(units in Pa)

| | % | % | % | | | | | | | | Std. | % |
|---|---|---|---|---|---|---|---|---|---|---|---|---|
| Form # | PVOH | SEPHA | POLAC | Run1 | Run 2 | Run 3 | Run 4 | Run 5 | Run 6 | Ave | Dev | RSD |
| 1 | 18.21% | 1.32% | 0.18% | 2950 | 3300 | 2950 | 2810 | 2800 | 2730 | 2923 | 204 | 7 |
| 2 | 18.99% | 3.19% | 0.18% | 5380 | 5770 | 5190 | 5120 | 5190 | 5620 | 5378 | 264 | 5 |
| 3 | 19.47% | 5.12% | 0.18% | 8650 | 9440 | 9470 | 9940 | 6940 | 5860 | 8383 | 1627 | 19 |
| 4 | 22.86% | 1.30% | 0.18% | 7830 | 7200 | 8090 | 8060 | 7500 | 7150 | 7638 | 417 | 5 |
| 5 | 23.65% | 3.28% | 0.18% | 13100 | 14200 | 14500 | 11500 | 15000 | 10100 | 13067 | 1915 | 15 |
| 6 | 23.35% | 5.07% | 0.18% | 24300 | 25500 | 24000 | 16700 | 21400 | 24500 | 22733 | 3256 | 14 |
| 7 | 27.27% | 1.27% | 0.17% | 11500 | 15700 | 12800 | 14100 | 16300 | 10100 | 13417 | 2410 | 18 |
| 8 | 27.27% | 3.13% | 0.17% | 28800 | 27400 | 23900 | 26000 | 21900 | 29000 | 26167 | 2823 | 11 |
| 9 | 27.87% | 4.00% | 0.18% | 39400 | 32900 | 31400 | 24500 | 26200 | 27000 | 30233 | 5515 | 18 |
| 10 | 18.81% | 0.00% | 0.18% | 3170 | 3590 | 3570 | 4210 | 3680 | 4320 | 3757 | 433 | 12 |
| 11 | 28.66% | 5.10% | 0.19% | — | 58800 | 38900 | — | — | — | 48850 | 14071 | 29 |

Viscosity of formulation before curing was measured on five gel solutions selected from the formulations of Table 4. POLACRILIN IRP 64 (designated POLAC in the Table) at 0.18 wt % was included in each. Viscosity could be measured using viscosity meters such as Haake RS 100 rheometer or RION Viscometer VT-04, made by Rion Company Ltd. The formulations and viscosity are listed in Table 5. The results in Table 5 showed that among the formulations in Table 5, which had about 1.3 wt % SEPHADEX™ QAE A-25 (designated as SEPHA in the Table), the ones with PVOH 10-98 were less viscous than that with PVOH 28-99 with the same PVOH content. However, the formulations when made into hydrogels had mechanical property suitable for fentanyl flux.

TABLE 5
Viscosity measurement of Hydrogel Solutions with SEPHADEX ™

| | | | | | | % Torque | |
|---|---|---|---|---|---|---|---|
| Formulation | Temp | RPM | Run 1 | Run 2 | Ave | Reading | |
| Formulation 1S | 40° C. | 5 | 3550 | 3170 | 3360 | 3.7 | 3.3 |
| PVOH 18.80% | | 10 | 3260 | 3170 | 3215 | 6.8 | 6.6 |
| SEPHADEX 1.29% | | 20 | 3240 | 3100 | 3170 | 13.5 | 12.9 |
| POLACRILIN 0.18% | 60° C. | 5 | 1630 | 1700 | 1665 | 13.6 | 14.2 |
| | | 10 | 1542 | 1578 | 1560 | 25.7 | 26.3 |
| | | 20 | 1506 | 1542 | 1524 | 50.2 | 51.4 |
| Formulation 2S | 40° C. | 5 | 7580 | 7390 | 7485 | 7.9 | 7.7 |
| PVOH 18.80% | | 10 | 7390 | 7340 | 7365 | 15.4 | 15.3 |
| Sephadex 1.29% | | 20 | 7300 | 7200 | 7250 | 30.4 | 30 |
| Polacrilin 0.18% | 60° C. | 5 | 3940 | 4030 | 3985 | 4.1 | 4.2 |
| | | 10 | 3650 | 3740 | 3695 | 7.6 | 7.8 |
| | | 20 | 3650 | 3790 | 3720 | 15.2 | 15.8 |
| Formulation 4S | 40° C. | 5 | 13060 | 13340 | 13200 | 13.6 | 13.9 |
| PVOH 18.80% | | 10 | 12530 | 12430 | 12480 | 26.1 | 25.9 |
| Sephadex 1.29% | | 20 | 12120 | 12070 | 12095 | 50.5 | 50.3 |
| Polacrilin 0.18% | 60° C. | 5 | 5950 | 6430 | 6190 | 6.2 | 6.7 |
| | | 10 | 5520 | 5950 | 5735 | 11.5 | 12.4 |
| | | 20 | 5500 | 5860 | 5680 | 22.9 | 24.4 |
| Formulation 5S | 40° C. | 5 | 40420 | 40220 | 40320 | 42.1 | 42 |
| PVOH 18.80% | | 10 | 38400 | 38260 | 38330 | 80 | 79.7 |
| Sephadex 1.29% | | 20 | — | — | — | — | — |
| Polacrilin 0.18% | 60° C. | 5 | 20540 | 19390 | 19965 | 21.4 | 20.2 |
| | | 10 | 18340 | 17230 | 17785 | 38.2 | 35.9 |
| | | 20 | 17350 | 17780 | 17565 | 72.3 | 74.1 |
| Formulation 10S | 40° C. | 5 | 22080 | 23040 | 22560 | 23 | 24 |
| PVOH 18.80% | | 10 | 21500 | 22460 | 21980 | 44.8 | 46.8 |
| Sephadex 1.29% | | 20 | 21360 | 22100 | 21730 | 89 | 92.1 |
| Polacrilin 0.18% | 60° C. | 5 | 10850 | 10660 | 10755 | 11.3 | 11.1 |
| | | 10 | 10460 | 10420 | 10440 | 21.8 | 21.7 |
| | | 20 | 10370 | 10390 | 10380 | 43.2 | 43.3 |

### Example 2 In Vitro Drug Flux

The method of iontophoretic transdermal flux in vitro measurement using separated human epidermis is well known in the art. The present measurements were made according to such prior known methods. Custom-built DELRON horizontal diffusion cells made in-house were used for all in vitro skin flux experiments. Anode with the same polarity as the drug is adhered to one end of the cell that functions as the donor cell. The counter electrode made of AgCl is adhered at the opposite end. These electrodes were connected to a current generator (Maccor) that applied a direct current across the cell. The Maccor unit was a device with in-built compliance voltage up to 20V to maintain constant iontophoretic current. For all in vitro electrotransport experiments, heat separated human epidermis was used. In a typical experiment, the epidermis was punched out into suitable circle ( 15/16 in, i.e., 2.4 cm diameter) and refrigerated just prior to use. The skin was placed on a screen ( 15/16 in) that fit into the midsection of the DELRON housing assembly. Underneath the screen was a small reservoir that was 0.5 in (1.25 cm) in diameter, 1/16 in (0.16 cm) deep and could hold approximately 250 μl of receptor solution. The stratum corneum side of the skin was placed facing the drug containing hydrogel (diameter 1.25 cm). The receptor solution (saline, phosphate or other buffered solutions compatible with the drug) was continuously pumped through the reservoir via polymer tubing (Upchurch Scientific) connected to the end of a syringe/pump assembly. The pump could be set to any desired flow rate. The drug containing reservoir was placed between the donor electrode and heat separated epidermis.

A custom-built DELRON spacer was used to encase the drug reservoir such that when the entire assembly was assembled together, the drug-containing gel was not pressed against the skin too hard as to puncture it. A number of spacers of varying thickness could be placed together using double-sided adhesives to accommodate polymer films of varying thickness or even multiple films. Double-sided adhesive was used to create a seal between all the DELRON parts and to ensure there were no leaks during the experiment. The entire assembly was placed between two heating blocks that were set at 34° C. to replicate skin temperature. The receptor solution was collected by the collection system, Hanson Research MICROETTE, interfaced to the experimental set up. The samples were collected from the reservoir underneath the skin directly into HPLC vials. The collection system was programmed to collect samples at specified time intervals depending on the length of the experiment, for example, at every hour for 24 hours. The Hanson system was designed such that it could collect from up to twelve cells. From the twelve cells, a piece of tubing takes the receptor solution to the MICROETTE and dispenses it into the HPLC vials loaded onto a rotating wheel that could hold up to 144 vials, or 12 vials for each cell. Once the vials on the wheel were filled, the vials could be replaced with empty vials to collect more samples. The samples could then be analyzed via HPLC to determine delivery efficiency of the drug in the formulation. A 1/10 diluted Delbeccos phosphate buffered saline (DPBS) receptor solution was used as the receiver fluid in vitro since it showed a good correlation of in vivo in vitro flux in the prior art. The buffer was pumped into the receptor solution reservoir at 1 ml/hr. The Hansen MICROETTE collection system could be programmed to collect periodically, e.g., every 1½ hour for 16 intervals over a 24 hour delivery experiment, or every 45 minutes for 12 hours, etc. The receptor solution flow could also be adjusted to higher or lower values. The cathodic hydrogel was similar to the anodic hydrogel except that it did not contain any drug or ion exchanger but contained saline.

Experiments were run on flux of fentanyl with fentanyl HCl in the gel having SEPHADEX™ QAE A-25 in the donor reservoir hydrogel. A design of experiments (DOE) was done to find the optimum SEPHADEX™ QAE loading in the donor reservoir hydrogel. The DOE table is Table 6. The % of IONSYS in the table refers to the fentanyl HCl loading as compared to the IONSYS fentanyl HCl loading in the donor reservoir, which had about 1.75 wt % fentanyl HCl. The PVOH gel contained 19 wt % of PVOH and 0.1 wt % of weak acidic cation exchanger POLACRILIN IRP 64 used for buffering effect for pH control.

TABLE 6
Fentanyl and SEPHADEX ™ Design

| | | wt % | | % |
|---|---|---|---|---|
| Form. | Design Model | Fentanyl HCl | % of IONSYS | SEPHADEX |
| DOE1 | −− | 0.696 | 40% | 0.65 |
| DOE2 | −0 | 0.696 | 40% | 1.30 |
| DOE3 | −+ | 0.696 | 40% | 1.95 |
| DOE4 | 0− | 1.044 | 60% | 0.65 |
| DOE5 | 00 | 1.044 | 60% | 1.30 |
| DOE6 | 0+ | 1.044 | 60% | 1.95 |
| DOE7 | +− | 1.392 | 80% | 0.65 |
| DOE8 | +0 | 1.392 | 80% | 1.30 |
| DOE9 | ++ | 1.392 | 80% | 1.95 |
| DOE10 | 00 | 1.044 | 60% | 1.30 |

The in vitro experimental result using heat separated epidermis are shown in TABLE 7. The experiments were done on skin donors A and B. The baseline experiments were done with fentanyl loaded hydrogel similar to the IONSYS fentanyl (i.e., 100% fentanyl loading without ion exchange resin).

TABLE 7
Flux data of Fentanyl & SEPHADEX ™ Design

| | | | Accumulative Flux | | | | | Residual |
|---|---|---|---|---|---|---|---|---|
| | | | (μg/cm²) | | | Steady State Flux | | Fentanyl at |
| | | Skin | After | After | After | Jss | Duration | 13.5 h |
| Form. | Model | Donor | 7.5 h | 13.5 h | 19.5 h | (μg/cm²h) | (h) | (mg/cm²) |
| 1 | −− | A | 312 | 745 | 942 | 71.2 | 6.5 | 0.45 |
| 2 | −0 | B | 512 | 977 | 1082 | 86.1 | 7.5 | 0.17 |
| 3 | −+ | A | 253 | 612 | 854 | 56.9 | 10.0 | 0.58 |
| 4 | 0− | B | 436 | 953 | 1333 | 78.2 | 15.0 | 0.78 |
| 5 | 00 | A | 340 | 816 | 1312 | 78.4 | 15.0 | 1.06 |
| 6 | 0+ | B | 385 | 832 | 1320 | 75.1 | 13.5 | 0.92 |
| 7 | +− | A | 324 | 711 | 1065 | 65.8 | 13.5 | 1.52 |
| 8 | +0 | B | 468 | 953 | 1402 | 80.3 | 16.1 | 1.37 |
| 9 | ++ | A | 359 | 801 | 1264 | 77.6 | 14.0 | 1.56 |
| 10 | 00 | B | 452 | 938 | 1363 | 89.1 | 9.4 | 0.87 |

| | | | | | | | |
|---|---|---|---|---|---|---|---|
| Baseline | A | 461 | 1007 | 1597 | 93.8 | 17.0 | 2.01 |
| Baseline | B | 504 | 1066 | 1558 | 90.7 | 18.0 | 2.17 |

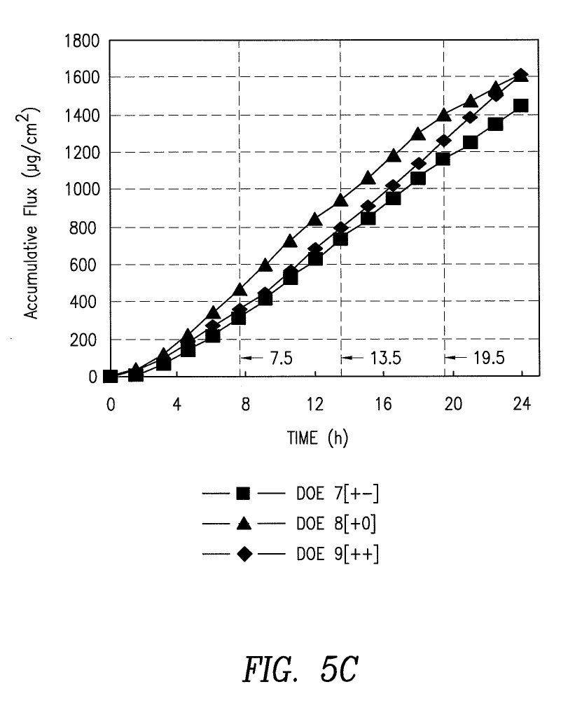

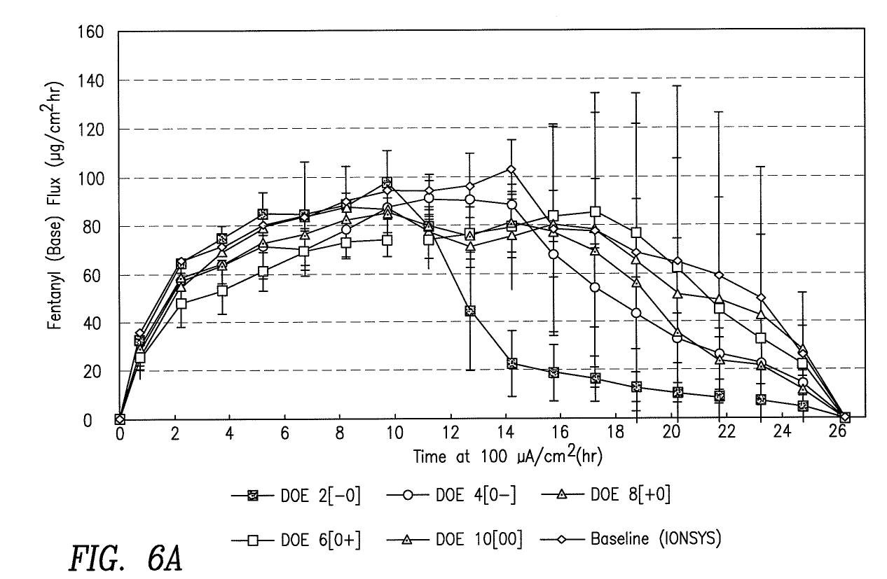

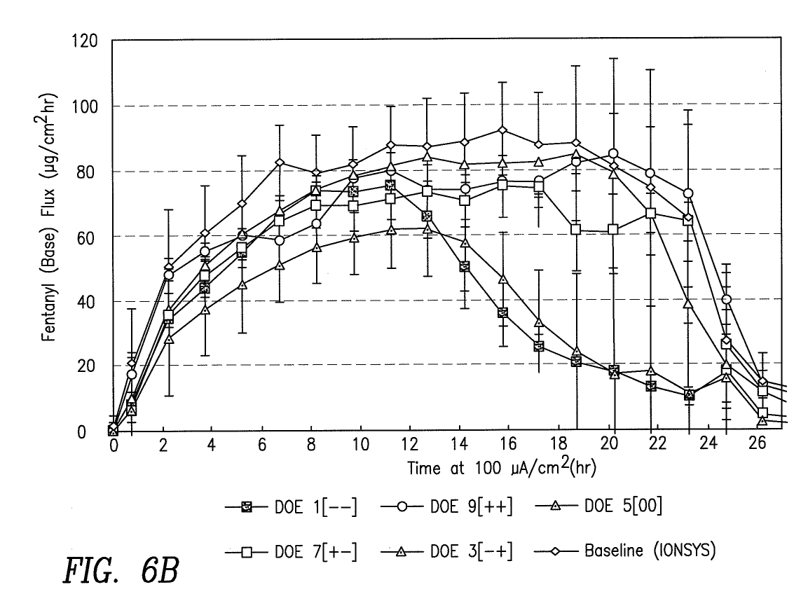

Table 7 shows the flux data using the gels of Table 6. Table 7 shows that the mid point loading level (1.3 wt %) of SEPHADEX™ corresponding to DOE 2, DOE 5, DOE 8 and DOE 10, had the highest area under the curve at 19.5 hour (AUC19.5) for each of the three fentanyl loading levels (0.70%, 1.04%, and 1.39%), compared with the higher or lower SEPHADEX™ loadings (0.65 wt % and 1.95 wt %). FIG. 5A to FIG. 5C show the accumulative fluxes for the DOE 1 to DOE 9 formulations. FIG. 6A shows the flux data versus time for the formulations applied to skin of Donor A of Table 7, whereas FIG. 6B shows the flux data versus time for the formulations applied to skin of Donor B of Table 7. In FIG. 6A, the shaded squares represent DOE2 (−0) data. The shaded circles represent DOE 4 (0−). The shade triangles represent DOE 8 (+0). The open squares represent DOE 6 (0+). The dotted triangles (triangle with a dot at the center) represent DOE 10 (00). The shaded diamonds represent Baseline IONSYS for comparison. FIG. 6A clearly shows that on skin Donor A, formulations (DOE 2, DOE 8, DOE 10) with mid level SEPHADEX™ content (1.3%) had good flux at the earlier part of the duration of the delivery. However, the flux DOE 2 (−0) fell after 12 hours because of fentanyl depletion. With mid level or high level of fentanyl, the formulations with mid level SEPHADEX™ resin content had AUC's larger than those with higher or lower level of SEPHADEX™ in 1 day.

In FIG. 6B (related to skin Donor B), the shaded squares represent DOE 1 (−−) data. The shaded circles represent DOE 9 (++). The shade triangles represent DOE 5 (00). The open squares represent DOE 7 (+−). The dotted triangles represent DOE 3 (−+). The shaded diamonds represent Baseline IONSYS. FIG. 6B shows that on skin Donor B, among the non-IONSYS formulations, the DOE having mid level SEPHADEX™ content (1.3%) had larger AUC than the DOE's with higher or lower SEPHADEX™ content. Overall, for 1-day delivery to skin Donor B, DOE 5 (00), which had the mid level of fentanyl concentration and mid SEPHADEX™ content (1.3%) had the largest AUC among the formulations with the new formulations (i.e., that were not IONSYS), i.e., larger than those of formulations having higher or lower fentanyl contents. The IONSYS formulations were provided only as control since they had more fentanyl than the new formulations tested. DOE 1, having only 40% the fentanyl content of IONSYS and low level SEPHADEX™, saw its flux fell quickly after 12 hours, due to fentanyl depletion.

Because in FIG. 6A and FIG. 6B the curves are very close together, the accumulative fluxes (area under the curves in FIG. 6) are easier to see by viewing FIG. 5A to FIG. 5C. FIG. 5A compares the accumulative flux of DOE 1, DOE 2 and DOE 3, all having 40% fentanyl loadings compared to the fentanyl loading in IONSYS system. The curve for DOE 2, which had 1.3 wt % SEPHADEX™ loading, had the overall best accumulative flux, essentially having the highest flux throughout the 24 hour period. FIG. 5B compares the accumulative flux of DOE 4, DOE 5 and DOE 6, all having 60% fentanyl loadings compared with the fentanyl loading in IONSYS system. The curve for DOE 5, which had 1.3 wt % SEPHADEX™ loading, had the overall best accumulative flux. FIG. 5C compares the accumulative flux of DOE 7, DOE 8 and DOE 9, all having 80% fentanyl loadings compared with the fentanyl loading in IONSYS system. The curve for DOE 8, which had 1.3 wt % SEPHADEX™ loading, had the overall best accumulative flux. Thus, these results indicated that the 1.30 w % is an optimum level of SEPHADEX™ for each of the respective fentanyl levels.

Separate experiments have shown that loadings above 2 wt % of SEPHADEX™ QAE ion exchanger result in lower cumulative release (presumably due to an increase in the viscosity of the hydrogel, increasing the resistance or “drag” of the gel on fentanyl ions) and those formulations without SEPHADEX™ anion exchanger delivered somewhat less drug than their counterparts with SEPHADEX™. The baseline experiments were done with donor reservoir at 100% drug loading as IONSYS without SEPHADEX™. The residual fentanyl numbers were the amount of fentanyl free base equivalent left after electrotransport. For comparison, the initial fentanyl (base equivalent) values were about 1.5 mg/cm2 for the 40% loading, 2.2 mg/cm2 for the 60% loading, 2.9 mg/cm2 for the 80% loading, and 3.6 mg/cm2 for the 100% loading.

By mathematically modeling and curve fitting the data of Table 7 and FIG. 5A to FIG. 6B, it was found that the optimal SEPHADEX™ QAE concentration for steady state flux Jₛₛ is about 1.3 wt % in the hydrogel, which contained about 19 wt % PVOH.

For the systems with 40% fentanyl loading (as compared to that of IONSYS), the slopes of the curves (FIG. 5A) started to fall after 13 hours, meaning that the fluxes were slowing due to fentanyl depletion. The systems with 60% and with 80% fentanyl loading (as compared to that of IONSYS) performed well up to about 20 hours. In fact, the slopes of the curves in FIG. 5B and FIG. 5C do not change much up to 24 hours, meaning that the fluxes were holding quite steady and that higher than 58% of fentanyl loading can be utilized without adverse effect.

Since the 80% fentanyl loading will have more % residual fentanyl than the 60% fentanyl loading, and the accumulative fluxes of FIG. 5B is close to those of FIG. 5C, the devices with 60% fentanyl loading had better % drug utilization than those with the 80% fentanyl loading. For 60% fentanyl loading (as compared to IONSYS), for the 1.3 wt % SEPHADEX™, the percent of fentanyl used (fluxed) were about 37% at 13.5 hours and about 60% at 19.5 hour. For 80% fentanyl loading, for the 1.95 wt % SEPHADEX™, the percent of fentanyl used (fluxed) was about 33% at 13.5 hours and about 48% at 19.5 hour. There were no signs of silver staining. Such systems could perform to achieve more than 50% or even more than 60% fentanyl utilization without observable staining. The results showed that the especially advantageous combination is 1.3 wt % SEPHADEX™ with 60% fentanyl loading (as compared to IONSYS) in the hydrogel. Within the ranges of fentanyl loading tested, the lower fentanyl loading resulted in higher utilization of fentanyl and in lower residual fentanyl. At 13.5 hours, the calculated amount of residual drug in this formulation was 0.17 mg/cm², compared with 2.17 mg/cm² for the baseline control and 1.06 mg/cm² for the 1.3 wt % SEPHADEX™ with 60% that of IONSYS. The lower fentanyl level formulations (40% of IONSYS) cannot maintain steady state flux beyond 13.5 hours due to drug depletion. However, such low fentanyl formulations would in fact be advantageous in a system requiring only 13.5 hours of operation or less (e.g., 10 or 12 hours) and resulting a small amount of residual fentanyl following use.

Experiments were run to test the capacity of the gels of the present invention to avoid silver staining. Table 8 showed the results. For the reservoirs with SEPHADEX™ ion exchangers, they contain double-layered gels (one layer with SEPHADEX™ ion exchangers positioned farther from skin and one layer without SEPHADEX™ ion exchangers nearer to the skin for a total thickness of 2.4 mm. The flux experimental conditions were as described above. In the Formulation Number A, the controls were fentanyl HCl gels like those used in IONSYS systems, i.e., they have 1.74 wt % fentanyl base equivalent loading and about 2.4 mm thick. The Formulation Number B controls were fentanyl HCl gels having about 60% of the fentanyl HCl loading as those of the 100% controls (i.e., 100% as compared to IONSYS fentanyl loading). The controls had no SEPHADEX™ resin. The Formulation Number C gels were gels made according the above process with 1.3 wt % SEPHADEX™ QAE A-25 in chloride form. The results show that the silver staining became observable in the 100% control gels after about 12 hours and only observable in the skin after 20 hours. In the 60% control gels, silver staining was seen after about 9 hours in the gels and seen in the skin after about 12 hours. In the SEPHADEX™ resins containing gels, silver staining was seen after 14 hours, whereas the skin did not show any silver staining even after 20 hours. This indicated that the SEPHADEX™ resins in chloride form protected the skin from silver staining even after 20 hour of iontophoretic drug delivery.

TABLE 8
Silver staining

| Formulation | | Ag migration | Ag migration (into |
|---|---|---|---|
| Number | Hydrogel Formulation | (into gel) | skin) |
| A | 100% control | >12 hr | >20 hr |
| B | 60% control | >9 hr | >12 hr |
| C | SEPHADEX | >14 hr | None (up to 20 |
| | coformulation | | hours) |

The above examples illustrate that donor reservoir with anion exchanger as anion source can reduce or prevent skin discoloration caused by silver ion migration. We found that MOWIOL 28-99 (“PVOH 28-99”) and MOWIOL 10-98 (“PVOH 10-98”) can be formulated with acceptable mechanical characteristics for anodic hydrogels with fentanyl HCl and SEPHADEX™ into hydrogels. It is therefore possible to make formulations with other PVOH grades to achieve similar characteristics for making anodic gels with fentanyl HCl and SEPHADEX™ to achieve acceptable flux without undue experimentation. Further, since SEPHADEX™ strong anion exchange resins are compatible with skin, hydrogels having SEPHADEX™ resin similar to those described above would not cause skin discoloration by erythema or edema. Considering the good performance of the gels with 60% fentanyl loading (compared to IONSYS), gels with even less than 60% fentanyl loading can be used for 20 hours without silver staining.

The above-described exemplary embodiments are intended to be illustrative in all respects, rather than restrictive, of the present invention. It is to be understood that various combinations and permutations of various parts and components of the schemes disclosed herein can be implemented by one skilled in the art without departing from the scope of the present invention.

We claim:

1. An electrotransport system for iontophoretic administration of a drug through a body surface of a patient, comprising:
(a) an anodic assembly having anodic electrode and an anodic reservoir, the anodic electrode having a sacrificial metal that generates metal ions in electrotransport, the anodic reservoir in electrical communication to said anodic electrode and comprising a cationic drug with an immobile biocompatible polysaccharide-based anion exchanger in the anodic reservoir, the anion exchanger having precipitate-forming anions that can react with the metal ion to form precipitate in the anodic reservoir thereby reducing migration of said metal ion to the body;
(b) a cathodic electrode assembly having a cathodic electrode in electrical communication with a cathodic reservoir; and
(c) circuitry electrically communicating with said anodic assembly and said cathodic assembly to drive electrotransport of said cationic drug.

2. The system of claim 1 wherein the sacrificial metal is silver, the precipitate forming anion is a halide, and the anion exchanger is a quaternary ammonium anion exchanger.

3. The system of claim 2 wherein the anion exchanger is dextran-based and has quaternary ammonium functionality having chloride as the halide.

4. The system of claim 2 wherein the cationic drug is fentanyl.

5. The system of claim 2 wherein the anodic reservoir is made with a carrier containing polyvinyl alcohol and the anion exchanger is dextran-based and has quaternary ammonium functionality having chloride as the halide.

6. The system of claim 2 wherein the anodic reservoir has anion exchanger that is cross-linked quaternary aminoethyl dextran and has quaternary ammonium functionality having chloride as the halide.

7. The system of claim 2 wherein the anodic reservoir contains fentanyl salt and the ion exchanger has an amount of halide ions such that the system can be operated for 20 hours without causing skin discoloration and after delivering a maximum amount of fentanyl the system is designed to deliver the amount of fentanyl remaining in the anodic reservoir is 40% or less of the fentanyl present before delivery.

8. The system of claim 2 wherein the anodic reservoir contains a hydrogel containing fentanyl hydrochloride and the system can deliver a flux of at least 60 μg/(cm²hr) fentanyl at 100 μA/cm² or more.

9. The system of claim 2 wherein the system can deliver the cationic drug effectively for at least 10 hours without staining the body surface.

10. The system of claim 2 wherein the anodic reservoir is made with a carrier containing polyvinyl alcohol and contains 1 wt % to 2 wt % of the anion exchanger, the anion exchange being cross-linked quaternary aminoethyl dextran with ionic capacity of 2.5-3.5 mmol/g on dry basis and containing quaternary ammonium functionality having chloride as the halide, wherein the system can be used for at least 20 hours without causing skin discoloration.

11. The system of claim 2 wherein the system contains less than 200 wt % of the maximum amount of cationic drug the device is designed to deliver.

12. The system of claim 2 wherein the anodic reservoir contains fentanyl salt and is made with a carrier containing polyvinyl alcohol and containing 1.3 wt % to 1.7 wt % of the anion exchanger, the anion exchanger being cross-linked quaternary aminoethyl dextran and containing quaternary ammonium functionality having chloride as the halide, wherein the system can be used for at least 20 hours without causing skin discoloration.

13. The system of claim 2 wherein the anodic reservoir contains a fentanyl salt and the system can be operated for 20 hours without causing skin discoloration and after delivering a maximum amount of fentanyl the system is designed to deliver the amount of fentanyl remaining in the anodic reservoir is 40% or less of the amount of fentanyl present before delivery.

14. The system of claim 1 wherein the anion exchanger is a strong anion exchanger and is dextran-based.

15. A method of preventing electrotransport discoloration of skin in iontophoretic delivery of a cationic drug, comprising:
applying an electrotransport device to the skin, the electrotransport device having an anodic reservoir and an anodic electrode, the anodic electrode having a sacrificial metal that generates metal ions in electrotransport, the anodic reservoir in electrical communication to said anodic electrode and comprising a cationic drug and having an immobile biocompatible polysaccharide-based anion exchanger in the anodic reservoir, the anionic exchanger having precipitate-forming anion that can react with the metal ion to form precipitate in the anodic reservoir thereby reducing migration of said metal ion to the body, the device having a maximum delivery amount of the cationic drug designed to be delivered that is more than 50% of the amount originally present before use;
using the device to deliver the cationic drug through the skin in an amount up to more than 50% of the amount originally present without discolorizing the skin and thereby rendering the device less subject to drug abuse of the cationic drug.

16. The method of claim 15 wherein the sacrificial metal is silver, the precipitate forming anion is a halide, and the anion exchanger is a quaternary ammonium anion exchanger.

17. The method of claim 16 wherein the anion exchanger is dextran-based and has quaternary ammonium functionality having chloride as the halide.

18. The method of claim 16 wherein the cationic drug is fentanyl.

19. The method of claim 16 wherein the anodic reservoir is made with a carrier containing polyvinyl alcohol and the anion exchanger is dextran-based and has quaternary ammonium functionality having chloride as the halide.

20. The method of claim 16 wherein the anodic reservoir has anion exchanger that is cross-linked quaternary aminoethyl dextran and has quaternary ammonium functionality having chloride as the halide.

21. The method of claim 16 wherein the anion exchanger contains an amount of halide ions at least stoichiometrically equivalent to silver ions that are to be produced by the anodic electrode during a predetermined period of delivery.

22. The method of claim 16 wherein the anodic reservoir contains a hydrogel containing fentanyl hydrochloride and the system can deliver a flux of at least 60 μg/(cm²hr) fentanyl at 100 μA/cm² or more.

23. The method of claim 16 wherein the system can deliver the cationic drug effectively for at least 20 hours without staining the body surface.

24. The method of claim 16 wherein the anodic reservoir is made with a carrier containing polyvinyl alcohol and contains 1 wt % to 2 wt % of the anion exchanger, the anion exchange being cross-linked quaternary aminoethyl dextran with ionic capacity of 2.5-3.5 mmol/g on dry basis and containing quaternary ammonium functionality having chloride as the halide, wherein the system can be used for at least 20 hours without causing skin discoloration.

25. The method of claim 16 wherein the system contains less than 200 wt % of the maximum amount of cationic drug it is designed to deliver.

26. The method of claim 16 wherein the anodic reservoir contains fentanyl salt and is made with a carrier containing polyvinyl alcohol and containing 1.3 wt % to 1.7 wt % of the anion exchanger, the anion exchanger being cross-linked quaternary aminoethyl dextran with ionic capacity of 2.5-3.5 mmol/g on dry basis and containing quaternary ammonium functionality having chloride as the halide, wherein the system can be used for at least 20 hours without causing skin discoloration.

27. The method of claim 16 wherein the anodic reservoir contains a fentanyl salt and the system can be operated for 20 hours without causing skin discoloration and after delivering a maximum amount of fentanyl designed to be delivered the amount of fentanyl remaining in the anodic reservoir is 40% or less of the amount of fentanyl originally present before use

28. A method of making an electrotransport drug delivery system for use on a body surface of a patient, comprising:
providing an anodic assembly having an anodic electrode and an anodic reservoir, the anodic electrode having a sacrificial metal that generates metal ion in electrotransport, the anodic reservoir in electrical communication to said anodic electrode and comprising a cationic drug and having an immobile biocompatible polysaccharide-based anion exchanger in the anodic reservoir, the anion exchanger having precipitate-forming anion that can react with the metal ion to form precipitate in the anodic reservoir thereby reducing migration of said metal ion to the body; and
connecting electrically said anodic assembly with a cathodic electrode assembly and a control circuitry, the cathodic assembly having a cathodic electrode in electrical communication with a cathodic reservoir, the control circuitry for controlling electrotransport of said cationic drug.

29. The method of claim 28 comprising dispersing the anion exchangers in the anodic reservoir and wherein the anion exchangers are insoluble.

30. A kit for administering a drug by iontophoresis transdermally through a body surface of a patient, comprising:
(a) an electrotransport device having an anodic electrode assembly, a cathodic electrode assembly, and control circuitry, the anodic assembly having an anodic electrode and an anodic reservoir comprising a cationic drug, the anodic electrode having a sacrificial metal that generates metal ions in electrotransport, the anodic reservoir in electrical communication to said anodic electrode and having an immobile biocompatible polysaccharide-based anion exchanger in the anodic reservoir, the anion exchanger having precipitate-forming anions that can react with the metal ion to form precipitate in the anodic reservoir thereby reducing migration of said metal ion to the body; the cathodic electrode assembly having a cathodic electrode in electrical communication with a cathodic reservoir; and the circuitry electrically communicating with said anodic assembly and cathodic assembly to drive electrotransport of said cationic drug transdermally; and
(b) an instruction print including instructions on electrotransport delivery of the drug up to a maximum amount, wherein the maximum amount is more than 50% the drug contained in the device before use.
